(19) United States
(12) Patent Application Publication
(10) Pub. No.: US 2015/0081029 A1
(43) Pub. Date: Mar. 19, 2015

(54) Advanced Methods, Techniques, Devices, and Systems for Cruciate Retaining Knee Implants

(71) Applicant: ConforMIS, Inc., Bedford, MA (US)

(72) Inventors: Raymond A. Bojarski, Attleboro, MA (US); Philipp Lang, Lexington, MA (US); John Slamin, Wrentham, MA (US); Terrance Wong, Needham, MA (US)

(21) Appl. No.: 14/389,987

(22) PCT Filed: Apr. 6, 2013
(86) PCT No.: PCT/US13/35536

§ 371 Date: Oct. 1, 2014

Related U.S. Application Data
(60) Provisional application No. 61/621,333, filed on Apr. 6, 2012.
(60) Provisional application No. 61/798,537, filed on Mar. 15, 2013.

(51) Int. Cl. A61B 17/16; A61F 2/38

(52) U.S. Cl. CPC A61B 17/1675; A61F 2/389; A61F 2240/00
USPC 623/20.32; 606/88; 29/407.04

(57) ABSTRACT

Improved implants, systems, tools and related methods for bi-cruciate retaining joint treatment are disclosed, including patient-adapted implants, systems, tools, and methods.

### RELATED APPLICATIONS

This application claims the benefit of U.S. Ser. No. 61/621,333, entitled “Advanced Methods, Techniques, Devices and Systems for Cruciate Retaining Knee Implants,” filed Apr. 6, 2012, and of U.S. Ser. No. 61/798,537, entitled “Advanced Methods, Techniques, Devices and Systems for Cruciate Retaining Knee Implants,” filed Mar. 15, 2013, the disclosure of each of which is hereby incorporated by reference herein in its entirety.

### TECHNICAL FIELD

The disclosure relates to improved and/or patient adapted (e.g., patient-specific and/or patient-engineered) orthopedic implants, as well as related methods, designs, systems and models. More specifically, disclosed herein are improved methods, designs and/or systems for joint implant components that facilitate retention and/or repair of connective and/or soft tissues during a joint replacement procedure.

### BACKGROUND

When a patient's knee is severely damaged, such as by osteoarthritis, rheumatoid arthritis, or post-traumatic arthritis, it may be desirous to repair and/or replace portions or the entirety of the knee with a total or partial knee replacement implant. Knee replacement surgery, also known as knee arthroplasty, can help relieve pain and restore function in injured and/or severely diseased knee joints, and is a well-tolerated and highly successful procedure. Where a total joint replacement is needed, it is often performed by a surgeon via an open procedure.

In an open procedure, the surgeon typically begins by making an incision through the various skin, fascia, and muscle layers to expose the knee joint and laterally dislocating the patella. The anterior cruciate ligament is often excised (if not already damaged or severed), and the surgeon will selectively sever or leave intact the posterior cruciate ligament—depending on the surgeon's preference and the condition of the PCL. Next, various surgical techniques are used to ablate, remove, shape or otherwise prepare the arthritic joint surfaces, and the tibia and femur are exposed for preparation and resection to accept various implant components.

Once the underlying bony anatomical support structures have been prepared, both the tibia and femur will typically receive an artificial joint component made of metal alloys, high-grade plastics and/or polymers to replace native anatomy and desirably function as a new knee joint. In the case of tibial implant components, the artificial joint can include a metal receiver tray that is firmly fixed to the tibia. In many cases, the tibial implant further includes a medical grade plastic insert (i.e. it may also be known as a “spacer”) that can be attached to the tray and positioned between the femoral component(s) and the tibial tray to create a smooth gliding surface for articulation of the components. Such a system can also allow for inserts of multiple sizes and/or thicknesses, which facilitates in-situ balancing of the knee as well as allowing the placement of inserts of differing designs and/or shapes.

Various surgical procedures in the past have sought to retain connective knee tissues during joint repair and/or replacement, but such techniques and associated implant designs have not gained widespread clinical acceptance for a variety of reasons. See, for example, U.S. Pat. Ser. No. 4,207,627 to Cloutier, entitled “Knee Prosthesis” filed Jun. 17, 1980, and J. M. Cloutier, Results of Total Knee Arthroplasty With A Non-Constrained Prosthesis, 65 J. BONE JOINT SURG. AM. 906 (1983); J. M. Cloutier et al., Total Knee Arthroplasty with Retention of Both Cruciate Ligaments: A Nine to Eleven-Year Follow-Up Study, 81-A J. BONE JOINT SURG. AM. 697 (May 1999).

While the implantation of total knee implant components via open procedures is a well accepted procedure that is well tolerated by patients and has a high success rate, surgeons often prefer to minimize the disruption and/or removal of hard and soft tissues except where absolutely necessary. For example, the use of minimally-invasive and/or less-invasive surgical procedures has become increasingly prevalent, as such procedures are often associated with faster patient healing times and less scarification of the patient's anatomy. Moreover, where portions of a patient's existing anatomy, such as an ACL or PCL, are substantially intact and/or functional in the damaged knee, many surgeons would prefer to maintain the integrity of these structures during the surgical implantation procedure, as such structures can greatly contribute to the ultimate stability and/or performance of the treated anatomy. Unfortunately, many current implant designs require the removal of such structures, even where such structures are fully functional, in order to accommodate the implant components.

Accordingly, there is a need in the art for patient-specific and/or patient-adapted joint replacement implant components and associated procedures that facilitate the retention and/or repair of anatomical structures such as the ACP and/or PCL (and/or other relevant hard and/or soft tissue structures) during surgical procedures. In addition, there is a need in the art for such implants and/or procedures that can be implanted via less-invasive and/or minimally-invasive procedures.

### SUMMARY

Various embodiments described herein include implant components suitable for use in a patient's knee, including multi-component systems incorporating one or more tibial trays, inserts, tools, methods, techniques and various devices that facilitate the preservation and/or repair of the ACL and PCL of a patient. Preservation of the ACL and/or PCL of a patient may improve physiological function and/or motion of the knee. Various other embodiments enable the retention of anatomical structures that can facilitate the surgical repair of various hard and/or soft tissues, including connective tissues such as the ACL and/or PCL of a patient.

In various embodiments, the implant components can include features such as cutout sections, notches or “windows” for accommodating various portions of the patient's natural anatomy, including bony anatomical structures and/or soft tissue structures. Optionally, these windows can facilitate the insertion, positioning and/or anchoring of the prosthesis to the underlying anatomical structures. In addition, various embodiments of tools and procedures described herein facilitate the preparation of the patient's anatomical structures for the implant components.

Disclosed herein are various advanced methods, devices, systems for implants, tools and techniques that facilitate the surgical repair of a knee joint while allowing retention of the natural central ligaments of the knee (and/or other related structures), thereby desirably preserving controlled rotation and translation of the repaired joint. In many embodiments, the procedures can provide adequate pain relief, preserve normal axial alignment of the limb, and preserve stability—this, in turn, will desirably reduce shear stresses at the component-cement-bone interfaces.

The embodiments described herein may be successfully applied to other damaged or diseased articulating joints where a surgeon desires to preserve natural ligaments and/or other underlying anatomical structures, including in the shoulder and/or hip. Also, various embodiments described herein can be successfully applied to total knee, bicompartmental or unicompartmental knee surgery.

Various embodiments described herein include systems having ligament retaining components and techniques, including: (1) tibial component systems; (2) improved femoral components; (3) surgical jigs/guides/tools; and (4) surgical methods/techniques.

### BRIEF DESCRIPTION OF THE DRAWINGS

The foregoing and other objects, aspects, features, and advantages of embodiments will become more apparent and may be better understood by referring to the following description, taken in conjunction with the accompanying drawings, in which:

FIG. 1 depicts a perspective view of a knee joint, showing associated hard tissue structures and soft connective tissues;

FIG. 2 depicts a frontal view of the femur and tibia bones of the knee joint of FIG. 1;

FIG. 3 depicts a frontal view of a tibia including a set of exemplary resection surfaces;

FIG. 4 depicts a perspective view of the tibia of FIG. 3, with a series of exemplary resections performed on the tibia and a retained central region;

FIG. 5 depicts a top plan view of one embodiment of a tibial implant component with an outer periphery depicted in dotted lines;

FIG. 6 depicts a top plan view of an alternate embodiment of a tibial implant component with an outer periphery depicted in dotted lines;

FIG. 7 depicts a perspective schematic view of one exemplary design for an embodiment of a tibial tray;

FIG. 8 depicts an alternative embodiment of a tibial tray for use with a PCL retaining implant system;

FIG. 9 depicts an alternative embodiment of a set of tibial tray components that have been selected and/or designed to accommodate the unique placement of a patient's PCL;

FIG. 10 depicts a top plan view of an unresected tibial surface, with an exemplary less-invasive and/or minimally-invasive surgical access window through the skin and overlying tissues;

FIG. 11 depicts a top plan view of one embodiment of a resected tibial surface, with various areas that may be difficult for a surgeon to access and/or visualize highlighted;

FIGS. 12 and 13 depict top plan and side perspective views of an alternate embodiment of a resected tibial surface including a central region having one or more canted or angled walls;

FIG. 14 depicts a schematic side view of a knee joint with a femur and tibia connected together via the ACL and PCL;

FIG. 15 depicts a schematic side view of the knee joint of FIG. 14, with the ACL severed or otherwise released and the tibia advanced relative to the femur for improved surgical access;

FIGS. 16 and 17 depict front and side views of one embodiment of a tibial guide tool or jig for preparing a tibia to receive a ligament sparing tibial tray;

FIG. 18 depicts the tibia of FIGS. 16 and 17 after removal of the jig, with three drill channels formed therein;

FIG. 19A depicts the tibia of FIG. 18, with various combinations of additional surgical steps performed to remove various sections of the tibial surface in preparation for the tibial tray implant;

FIG. 19B depicts a frontal perspective view of the tibia of FIG. 19A, with portions of the drill channels forming sections of the prepared tibial surface;

FIGS. 19C and 19D depict front plan views of a drill channel and exemplary cut planes;

FIG. 20 depicts a top plan view of one embodiment of a tibial tray for use in an ACL/PCL retention procedure;

FIG. 21 depicts a top plan view of an alternate embodiment of a tibial tray, including a pair of attachment or locking mechanisms for securing one or more inserts to the tray;

FIG. 22 depicts a diagram of a tibial surface prepared in accordance with various embodiments described herein;

FIG. 23 depicts a top plan view of an alternate embodiment of a tibial tray design that incorporates a notched section to accommodates a remaining natural section of the tibial surface;

FIG. 24 depicts a top plan view of alternative designs for a tibial tray, including exemplary medial and lateral articulating surfaces;

FIG. 25 depicts an alternate embodiment of a tibial tray design, including various rounded or curved surfaces;

FIG. 26 depicts an alternate embodiment of a tibial tray design, including one or more flattened and/or angled surfaces;

FIGS. 27 and 28 depict top plan and side views of an alternative embodiment of a surgical cut or guide tool for use in preparing portions of the surface of the tibial bone;

FIG. 29 depicts a side plan view of an alternate embodiment of a tibial guide tool or jig for use in preparing the surface of a tibia;

FIG. 30 depicts a side plan view of another alternate embodiment of a tibial guide tool or jig for use in preparing the surface of a tibia;

FIG. 31 depicts an alternate embodiment of a jig having external indicia that substantially matches or indicates one or more features of the targeted anatomy and/or features of interest for the surgeon's reference;

FIGS. 32 and 33 depict a set of jigs for use in creating a plurality of cut planes and/or other surgical objectives;

FIG. 34 depicts a side plan view of an alternate embodiment of a tibial guide tool or jig for use in preparing the surface of a tibia;

FIGS. 35 and 36 depict exemplary surgical cutting instruments;

FIG. 37 shows an image of a bi-cruciate retaining patient-adapted knee replacement implant system including a patient-specific femoral component and a patient-specific cruciate-retaining tibial tray component;

FIGS. 38A through 38C depict three different types of step cuts separating medial and lateral resection cut facets on a patient's proximal tibia;

FIGS. 39A through 39D depict side views of a tibial plateau in an uncut condition and implanted with various combinations of lower metal backed tibial tray components and upper inserts;

FIGS. 40A through 40E show side views of exemplary combinations of tibial tray and insert designs;

FIG. 41 is a flow chart for adapting a blank implant component for a particular patient;

FIG. 42 shows an example of a femoral component design for a bi-cruciate retaining knee implant system as described herein.

### TIBIAL COMPONENT SYSTEMS

Tibial component systems embodiments described herein facilitate the design of “patient-specific,” “patient-engineered” and/or “standard off-the-shelf” tibial trays and tibial inserts (and various combinations thereof) that preserve one or both natural central ligaments of the knee (including the ACL and PCL). Such systems can significantly reduce the potential for migration, instability, and preserve the normal flexion, extension, and rotation of the knee.

In various embodiments, the size of the ligament preserving tibial tray may be designed as patient-specific or patient-engineered by incorporating patient-specific and/or patient-engineered measurements into the outer perimeter of the tibial component. The patient image data (as well as data derived from patient-specific data, including patient-engineered data) can be used to specifically design the outer perimeter of the tibial tray and its internal structures to create a unique patient-specific size and shape for the patient. In addition, a database of patient image data may be evaluated and statistically analyzed to create several standard “blank” sizes to be available for use with most common patients. The standard “blank” sizes may be kept in inventory until needed, and then modified (if and as necessary) and shipped for a scheduled surgery. Other outer perimeter embodiments may comprise shapes that may incorporate symmetric or asymmetric medial and lateral sides, may include offset medial and lateral sides and/or may include oblique symmetric or asymmetric medial and lateral sides. Other shapes may incorporate a one-piece design, a two-piece design, or a modular design.

In other embodiments, a ligament retaining tibial tray component may be designed specifically to include central ligament preservation features. The tibial tray may have a variety of unique internal or peri-ligament area shapes to accommodate one or both central ligaments in the knee. The shapes within the tibial peri-ligament area in the tray may comprise of shapes similar to “W,” “V”, “H.” Each of these shapes may be designed to accommodate the angular or oblique nature of the ligaments. Also, each of these shapes may involve a combination of “W,” “V”, “H” with the various outer perimeter embodiments described above. In addition, the peri-ligament area shapes may have shapes that are trapezoidal, triangular, square, pentagon, octagon, and other similar shapes within this groove.

In various embodiments, the peri-ligament area shapes can be patient specific, pre-configured and/or standard off-the-shelf, for example, in two or three different geometries or size. Optionally, a user or a computer program can have a library of CAD files or subroutines with different sizes, shapes, perimeter and geometries to be made available. Moreover, the type of cruciate retaining tibial tray (i.e. one-piece v. two-piece design) can be selected based on patient specific parameters, e.g. body weight, height, gender, race, activity level etc.), and may include one or more combinations of peri-ligament area shapes.

In various embodiments, the tibial tray cavities (i.e. they are also known as tibial tray receptacles) can be designed to receive one or more tibial inserts (or other quantities, as desired). The tibial tray may have patient specific cavity dimensions or a combination thereof. The once-piece or the two-piece cavity designs may include the ability to snap fit, press fit, or have an improved mechanical fixation for the tibial insert. The two-piece design may include features that provide easy guidance to place the inserts into the cavities for accurate orientation and placement of the insert. Also, both the one-piece and two-piece designs may also have audible signals or other indicators that can notify the surgeon that the insert is firmly fixed to the tray. In alternative embodiments, the tibial tray cavities can be prepared in multiple sizes, e.g., having various AP dimensions, ML dimensions, and/or stem and keel dimensions and configurations. However, in other-sized embodiments (e.g., having larger or small tray ML and/or AP dimensions), the stem and keel can be larger, smaller, or have a different configuration.

In other embodiments of the tibial tray cavities, the cavities can be designed to include permanent fixation of the tibial inserts or provide a mechanism for release of the insert. Permanent fixation may be accomplished by attaching the insert to the tray using mechanical means or the insert may be overmolded with the tray to create an assembly of the tray and the insert together. In an alternative design, the tray cavities may be designed to include one or more quick-release mechanisms to release the insert for insert size/thickness interchangeability. In various embodiments, the tibial tray may be designed to have a release mechanism that requires an additional tool so as to prevent or limit inadvertent release of the implant (or where the insert may be semi-permanent and/or require subsequent removal).

In other embodiments, the tibial tray cavities are designed to accept a tibial insert. The tibial insert may be designed as one-piece, two-piece, patient-specific, or a combination thereof, and there may be one or more cavities formed into a given tibial tray. For example, a tibial insert may use a patient-adapted profile to substantially match the profile of the patient's resected tibial surface. More specifically, the insert can be designed to match or optimize one or more patient-specific features based on patient-specific data, such as a patient-specific perimeter profile and/or one or more medial coronal, medial sagittal, lateral coronal, lateral sagittal bone-facing insert curvatures. The insert may be perimeter-matched to some or all of the tibial tray. In alternative embodiments, the tray perimeter may be undersized or the perimeter modified a desired amount to allow some rotation of the tray by the physician without significant overhang off the resected tibial surface. Similar over-sizing of the peri-ligament area may be utilized to allow for some rotation of the tray by the physician without significant interference from the tibial structures within the peri-ligament area.

In addition, the tibial inserts may also be designed to accommodate the pen-ligament area of the tibial tray. The tibial inserts may be designed to similar shapes as described above for the tibial tray peri-ligament area, or the tibial inserts may include features that provide ligament reliefs or ligament “guides” to prevent and/or limit unwanted contact, inflammation or “wear and tear.” This may include extreme bevels, chamfers, or angled edges to reduce wear or contact for potential inflammation of the PCL and/or other soft tissue structures.

The tibial insert may also be uniquely designed to accommodate the locking mechanisms designed in the cruciate retaining tibial tray. The locking mechanism may be selected and/or designed to desirably avoid or limit compromise of the retained ligaments and facilitate ease of use by the surgeon. In various alternative embodiments, a tibial insert may be designed to incorporate an integrally-formed tab or other feature that engages into the locking mechanism to reduce or eliminate motion or rotation to reduce the potential for subsequent failure of the knee implant. The tibial insert may also have other constructs to engage with the locking mechanism (i.e. detents, tubes, screw attachments, etc.).

Improved Cruciate Retaining Femoral Component

The femoral component is another important aspect of knee surgery, and the femoral component will desirably include features that accommodate cruciate retention in the knee. The femoral component may be designed as patient-specific or patient-engineered by incorporating patient-specific and/or patient-engineered measurements into the femoral component. The patient image data (as well as data derived from patient-specific data, including patient-engineered data) can be used to specifically design femoral component(s) to create a unique patient-specific size and shape for the patient.

In alternative embodiments, the patient image data may be used to design femoral components that have asymmetric or symmetric medial or lateral sides due to the positioning of one or both cruciate ligaments. The medial or lateral sides may also have different AP or ML dimensions. Also, the condylar groove may also be designed to have a deeper/larger cut, have a variety of shapes, may be obliquely cut, or be a combination of one or more of these shapes and/or designs.

In other embodiments, the femoral component used in unicompartmental or bicompartmental surgeries may be used in combination with the improved cruciate retaining tibial tray component system designs as described above. In an alternative embodiment, the femoral component may also be a one-piece or two-piece design. For example, the two-piece design could include insertion of a unicompartmental femoral component with a uniquely designed 2^{nd} piece, including another unicompartmental or bicompartmental femoral implant(s) to accommodate the reduced area and space when preserving one or both ligaments.

Cruciate Retaining Surgical Jigs/Guides/Resection Tools

A variety of traditional guide/jigs/resection tools are available to assist surgeons in preparing a joint for an implant, for example, for resectioning one or more of a patient's biological structures during a joint implant procedure. However, these traditional guide tools typically are not designed to match the shape (contour) of a particular patient's biological structure(s). Moreover, these traditional guide tools typically are not designed to impart patient-optimized placement for the resection cuts, and are not designed to accommodate the reduced space when preserving one or more cruciate ligaments. Thus, using and properly aligning traditional guide tools, as well as properly aligning a patient's limb (e.g., in rotational alignment, in varus or valgus alignment, or alignment in another dimension) in order to orient these traditional guide tools, can be an imprecise and complicated part of the implant procedure. As used herein, “jig” also can refer to guide tools, for example, to guide tools that guide resectioning of a patient's biological structure. As a result, certain embodiments described herein provide improved surgical guide jigs/guides/tools for preparing a patient's biological structure during a cruciate retaining and/or repairing joint implant procedure.

In certain embodiments, a guide tool includes at least one feature for directing a surgical instrument to deliver a patient-engineered, patient-specific or standard feature(s) to the patient's biological structure, for example, a resected hole or a resection cut for engaging a patient-engineered implant peg or a patient-engineered implant bone-facing surface. In addition to the patient-engineered feature, in certain embodiments one or more of the guide tool's bone-facing surfaces can be designed to be patient-specific so that it substantially negatively-matches a portion of the patient's joint surface. In addition or alternatively, one or more of the guide tool's bone-facing surfaces can be standard in shape.

The guide/jigs/resection tools further can include at least one aperture for directing movement of a surgical instrument, for example, a securing pin or a cutting tool. One or more of the apertures can be designed to guide the surgical instrument to deliver a patient-optimized placement for, for example, one or more securing pins or resection cuts. In addition or alternatively, one or more of the apertures can be designed to guide the surgical instrument to deliver a standard placement for, for example, for one or more securing pins or resection cuts. Alternatively, certain guide tools can be used for purposes other than guiding a drill or cutting tool. For example, balancing and trial guide tools can be used to assess knee alignment and/or fit of one or more implant components or inserts. Also, the balancing and trial guide tools can be used in combination with other jigs to deliver a more accurate or precise resected surface of the bone.

The guide tools described herein can include any combination of patient-specific features, patient-engineered features, and/or standard features. For example, a patient-specific guide tool can include at least one feature that is preoperatively designed and/or selected to substantially match one or more of the patient's biological features. A standard guide tool can include at least one feature that is selected from among a family of limited options, for example, selected from among a family of 5, 6, 7, 8, 9, or 10 options. Moreover, in certain embodiments a set or kit of guide tools is provided in which certain guide tools in the set or kit include patient-specific, patient-engineered and/or standard features.

Information regarding the misalignment and the proper mechanical alignment of a patient's limb can be used to preoperatively design and/or select one or more features of a joint implant and/or implant procedure. For example, based on the difference between the patient's misalignment and the proper mechanical axis, a knee implant and implant procedure can be designed and/or selected preoperatively to include implant and/or resection dimensions that substantially realign the patient's limb to correct or improve a patient's alignment deformity. In addition, the process can include selecting and/or designing one or more surgical tools (e.g., guide tools or cutting jigs) to direct the clinician in resectioning the patient's bone in accordance with the preoperatively designed and/or selected resection dimensions.

In certain embodiments, the degree of deformity correction that is necessary to establish a desired limb alignment is calculated based on information from the alignment of a virtual model of a patient's limb. The virtual model can be generated from patient-specific data, such 2D and/or 3D imaging data of the patient's limb. The deformity correction can correct varus or valgus alignment or antecurvatum or recurvatum alignment. In various embodiments, the desired deformity correction returns the leg to normal alignment, for example, a zero degree biomechanical axis in the coronal plane and absence of genu antecurvatum and recurvatum in the sagittal plane.

The preoperatively designed and/or selected implant or implant component, resection dimension(s), and/or cutting jig(s) can be employed to correct a patient's alignment deformity in a single plane, for example, in the coronal plane or in the sagittal plane, in multiple planes, for example, in the coronal and sagittal planes, and/or in three dimensions. In one embodiment, where the patient's lower limb is misaligned in the coronal plane, for example, a valgus or varus deformity, the deformity correction can be achieved by designing and/or selecting one or more of a resection dimension, an implant component thickness, and an implant component surface curvature that adjusts the mechanical axis or axes into alignment in one or more planes. For example, a lower limb misalignment can be corrected in a knee replacement by designing or selecting one or more of a femoral resection dimension, a femoral implant component thickness, a femoral implant component surface curvature, a tibial resection dimension, a tibial implant component thickness, a tibial implant component insert thickness, and a tibial implant component surface curvature to adjust the femoral mechanical axis and tibial mechanical axis into alignment in the coronal plane.

In certain embodiments, bone cuts and implant shape including at least one of a bone-facing or a joint-facing surface of the implant can be designed or selected to achieve normal joint kinematics.

In certain embodiments, a computer program simulating biomotion of one or more joints, such as, for example, a knee joint, or a knee and ankle joint, or a hip, knee and/or ankle joint can be utilized. In certain embodiments, patient-specific imaging data can be fed into this computer program. For example, a series of two-dimensional images of a patient's knee joint or a three-dimensional representation of a patient's knee joint can be entered into the program. Additionally, two-dimensional images or a three-dimensional representation of the patient's ankle joint and/or hip joint may be added.

Alternatively, patient-specific kinematic data, for example obtained in a gait lab, can be fed into the computer program. Alternatively, patient-specific navigation data, for example generated using a surgical navigation system, image guided or non-image guided can be fed into the computer program. This kinematic or navigation data can, for example, be generated by applying optical or RF markers to the limb and by registering the markers and then measuring limb movements, for example, flexion, extension, abduction, adduction, rotation, and other limb movements.

Optionally, other data including anthropometric data may be added for each patient. These data can include but are not limited to the patient's age, gender, weight, height, size, body mass index, and race. Desired limb alignment and/or deformity correction can be added into the model. The position of bone cuts on one or more articular surfaces as well as the intended location of implant bearing surfaces on one or more articular surfaces can be entered into the model.

A patient-specific biomotion model can be derived that includes combinations of parameters listed herein. The biomotion model can simulate various activities of daily life including normal gait, stair climbing, descending stairs, running, kneeling, squatting, sitting and any other physical activity. The biomotion model can start out with standardized activities, typically derived from reference databases. These reference databases can be, for example, generated using biomotion measurements using force plates and motion trackers using radiofrequency or optical markers and video equipment.

The biomotion model can then be individualized with use of patient-specific information including at least one of, but not limited to the patient's age, gender, weight, height, body mass index, and race, the desired limb alignment or deformity correction, and the patient's imaging data, for example, a series of two-dimensional images or a three-dimensional representation of the joint for which surgery is contemplated.

An implant shape including associated bone cuts generated in the preceding optimizations, for example, limb alignment, deformity correction, bone preservation on one or more articular surfaces, can be introduced into the model. Many exemplary parameters can be measured in a patient-specific biomotion model.

### Parameters Measured in a Patient-Specific Biomotion Model for Various Implants

| Joint implant | Measured Parameter |
|---|---|
| knee | Medial femoral rollback during flexion |
| knee | Lateral femoral rollback during flexion |
| knee | Patellar position, medial, lateral, superior, inferior for different flexion |
| | and extension angles |
| knee | Internal and external rotation of one or more femoral condyles |
| knee | Internal and external rotation of the tibia |
| knee | Flexion and extension angles of one or more articular surfaces |
| knee | Anterior slide and posterior slide of at least one of the medial and |
| | lateral femoral condyles during flexion or extension |
| knee | Medial and lateral laxity throughout the range of motion |
| knee | Contact pressure or forces on at least one or more articular surfaces, |
| | e.g. a femoral condyle and a tibial plateau, a trochlea and a patella |
| knee | Contact area on at least one or more articular surfaces, e.g. a femoral |
| | condyle and a tibial plateau, a trochlea and a patella |
| knee | Forces between the bone-facing surface of the implant, an optional |
| | cement interface and the adjacent bone or bone marrow, measured at |
| | least one or multiple bone cut or bone-facing surface of the implant on |
| | at least one or multiple articular surfaces or implant components. |
| knee | Ligament location, e.g. ACL, PCL, MCL, LCL, retinacula, joint |
| | capsule, estimated or derived, for example using an imaging test. |
| knee | Ligament tension, strain, shear force, estimated failure forces, loads |
| | for example for different angles of flexion, extension, rotation, |
| | abduction, adduction, with the different positions or movements |
| | optionally simulated in a virtual environment. |
| knee | Potential implant impingement on other articular structures, e.g. in |
| | high flexion, high extension, internal or external rotation, abduction or |
| | adduction or any combinations thereof or other angles/positions/ |
| | movements. |
| Hip, shoulder or | Internal and external rotation of one or more articular surfaces |
| other joint | |
| Hip, shoulder or | Flexion and extension angles of one or more articular surfaces |
| other joint | |
| Hip, shoulder or | Anterior slide and posterior slide of at least one or more articular |
| other joint | surfaces during flexion or extension, abduction or adduction, |
| | elevation, internal or external rotation |
| Hip, shoulder or | Joint laxity throughout the range of motion |
| other joint | |
| Hip, shoulder or | Contact pressure or forces on at least one or more articular surfaces, |
| other joint | e.g. an acetabulum and a femoral head, a glenoid and a humeral head |
| Hip, shoulder or | Forces between the bone-facing surface of the implant, an optional |
| other joint | cement interface and the adjacent bone or bone marrow, measured at |
| | least one or multiple bone cut or bone-facing surface of the implant on |
| | at least one or multiple articular surfaces or implant components. |
| Hip, shoulder or | Ligament location, e.g. transverse ligament, glenohumeral ligaments, |
| other joint | retinacula, joint capsule, estimated or derived, for example using an |
| | imaging test. |
| Hip, shoulder or | Ligament tension, strain, shear force, estimated failure forces, loads |
| other joint | for example for different angles of flexion, extension, rotation, |
| | abduction, adduction, with the different positions or movements |
| | optionally simulated in a virtual environment. |
| Hip, shoulder or | Potential implant impingement on other articular structures, e.g. in |
| other joint | high flexion, high extension, internal or external rotation, abduction or |
| | adduction or elevation or any combinations thereof or other angles/ |
| | positions/movements. |

The above list is not meant to be exhaustive, but only exemplary. Any other biomechanical parameter known in the art can be included in the analysis.

The resultant biomotion data can be used to further optimize the implant design with the objective to establish normal or near normal kinematics. The implant optimizations can include one or multiple implant components. Implant optimizations based on patient-specific data including image based biomotion data include, but are not limited to:
Changes to external, joint-facing implant shape in coronal plane
Changes to external, joint-facing implant shape in sagittal plane
Changes to external, joint-facing implant shape in axial plane
Changes to external, joint-facing implant shape in multiple planes or three dimensions
Changes to internal, bone-facing implant shape in coronal plane
Changes to internal, bone-facing implant shape in sagittal plane
Changes to internal, bone-facing implant shape in axial plane
Changes to internal, bone-facing implant shape in multiple planes or three dimensions
Changes to perimeter of implant shape in coronal plane
Changes to perimeter of implant shape in sagittal plane
Changes to perimeter of implant shape in axial plane
Changes to perimeter of implant shape in multiple planes or three dimensions
Changes to implant notch shape in coronal plane
Changes to implant notch shape in sagittal plane
Changes to implant notch shape in axial plane
Changes to implant notch shape in multiple planes or three dimensions
Changes to one or more bone cuts, for example with regard to depth of cut, orientation of cut

Various embodiments contemplate any single one or combinations of the above or all of the above on at least one articular surface or implant component or multiple articular surfaces or implant components.

When changes are made on multiple articular surfaces or implant components, these can be made in reference to or linked to each other. For example, in the knee, a change made to a femoral bone cut based on patient-specific biomotion data can be referenced to or linked with a concomitant change to a bone cut on an opposing tibial surface, for example, if less femoral bone is resected, the computer program may elect to resect more tibial bone.

Similarly, if a femoral implant shape is changed, for example on an external surface, this can be accompanied by a change in the tibial component shape. This is, for example, particularly applicable when at least portions of the tibial bearing surface negatively-match the femoral joint-facing surface.

Similarly, if the footprint of a femoral implant is broadened, this can be accompanied by a widening of the bearing surface of a tibial component. Similarly, if a tibial implant shape is changed, for example on an external surface, this can be accompanied by a change in the femoral component shape. This is, for example, particularly applicable when at least portions of the femoral bearing surface negatively-match the tibial joint-facing surface.

Similarly, if a patellar component radius is widened, this can be accompanied by a widening of an opposing trochlear bearing surface radius, or vice-versa.

Cruciate Retaining Surgical Methods/Techniques

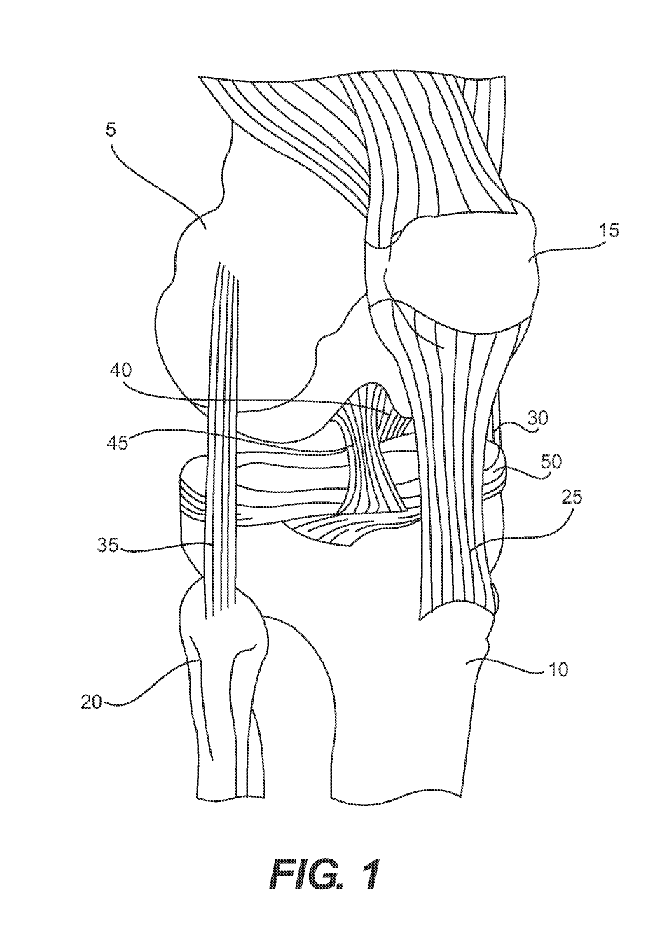

FIG. 1 depicts a perspective view of a knee joint, showing a femur 5, a tibia 10, a patella 15 and a fibula 20. A number of connective structures extend between the various bones and/or other structures of the knee, including the patellar tendon 25, the medial collateral ligament 30 (MCL), the lateral collateral ligament 35 (LCL), the posterior cruciate ligament 40 (PCL) and the anterior cruciate ligament 45 (ACL). Also shown is the meniscus 50, which is depicted between the femur 5 and the tibia 10.

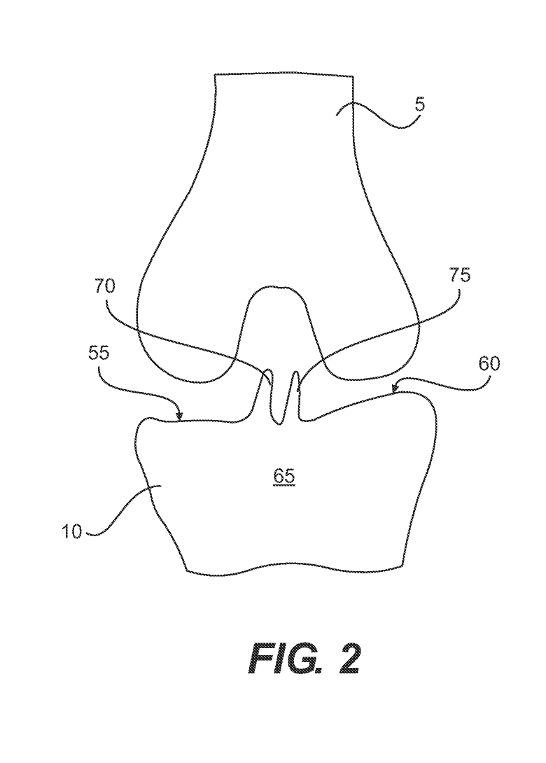

FIG. 2 depicts a frontal view of the femur 5 and tibia 10 of the knee joint of FIG. 1, the tibial surface including a medial surface 60, a lateral surface 55 and a central region 65 which includes a medial intercondylar tubercle 75 and a lateral intercondylar tubercle 70.

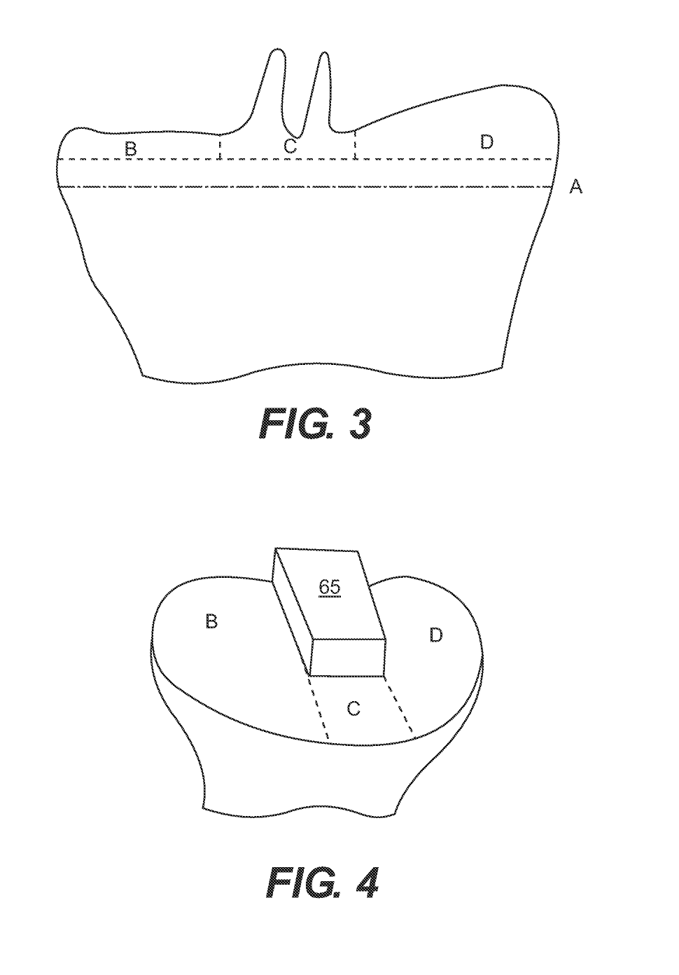

FIG. 3 depicts a frontal view of a tibia including a series of resection surfaces A, B, C and D. Traditionally, a single planar resection of the entire tibia is performed, thereby creating a flat planar surface for placement of tibial components (not shown). Alternatively, a resection of portions B and/or D is performed to accommodate unicoldylar and/or bicondylar replacement/resurfacing of individual articulating surfaces of the tibia. In various embodiments described herein, preparation of the tibial surface can include removal of material from multiple regions of the tibia, including, for example, B, D, and some or all portions of C. If desired, the depth of the various tibial resections can be varied, and can include depths less than, equal to, or greater than those shown (i.e., A, B, C and/or D) on the figure. In addition, resection depths and/or angulations can vary across the tibia, as will be described herein.

FIG. 4 depicts a perspective view of the tibia of FIG. 3, with resection surfaces B, C and D performed on the tibia. In this embodiment, a portion of the central region 65 has been retained, which desirably allows retention of the patient's ACL and PCL during and subsequent to the knee replacement surgery. In alternate embodiments, the retention of the central region 65 can provide an anchoring location for repair of one or more central ligaments using, for example, a tibial tunneling and ligament anchoring technique.

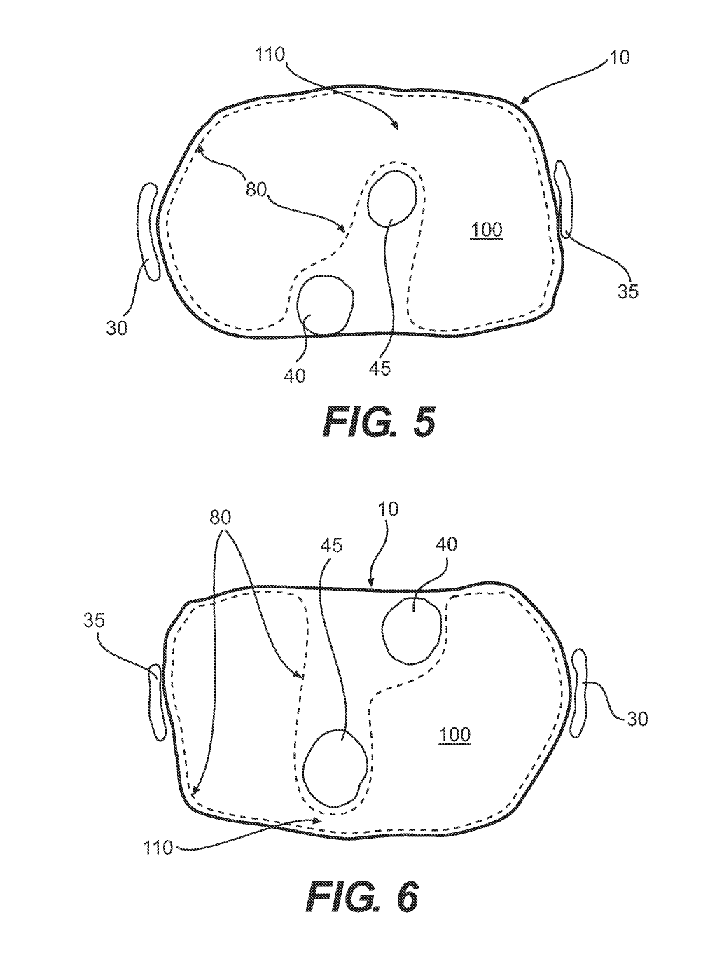

FIGS. 5 and 6 depict top plan views of exemplary tibial surfaces 10 and relevant anatomy showing exemplary tibial implant components 100 with an outer periphery depicted in dotted lines 80. As can be seen in these figures, the tibial implant components 100 cover a significant portion of the surface of the tibia 10, but include one or more peri-ligament areas, hereinafter referred to as notches or “cut out” sections, that desirably accommodate one or more regions of the tibial surface where the ACL 45 and PCL 40 connect to the tibia 10. Comparison of the components 100 of FIGS. 5 and 6 show, among other differences, a difference in anterior/posterior width of an anterior bridge 110. Accordingly, in various embodiments, the design of the perimeter and/or other features of the tibial tray can be, at least in part, dependent upon the specific patient anatomy.

In various embodiments, the use of patient-specific image data, either alone or in combination with patient-engineered and/or standard data, can allow a physician and/or implant designer to design and/or select an implant appropriate to the patient's specific condition. For example, patient specific image data may be utilized to determine the location, orientation and/or condition of anatomical structures such as the ACL and/or PCL, including the attachment locations and supporting structures for such ligaments. Using this data, one or more implant components can be selected and/or designed to resurface and/or replace damaged or diseased articulating surfaces while avoiding the ACL and/or PCL or other connective or soft tissue structures. In a similar manner, the outer perimeter of the tray proximate other structures, such as, for example, the MCL and LCL, can be designed to accommodate, avoid, encompass and/or otherwise account for the presence of such anatomical structures.

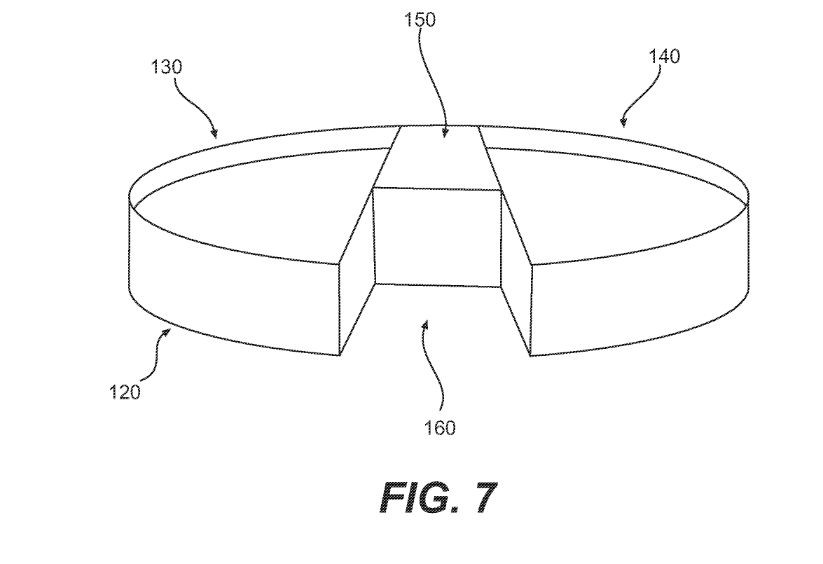

FIG. 7 depicts a perspective schematic view of one exemplary design for a tibial tray 120. In this embodiment, the tray 120 includes a lateral tray portion 140, a medial tray portion 130 and an anterior bridge portion 150 connecting the lateral and medial tray portions. A notch section 160 is formed in a posterior portion of the tray 120. Desirably, the notch section 160 is sized and configured to accommodate a central region (e.g., central region 65, illustrated in FIG. 4) of a tibia that has been prepared for implantation of the tray 120.

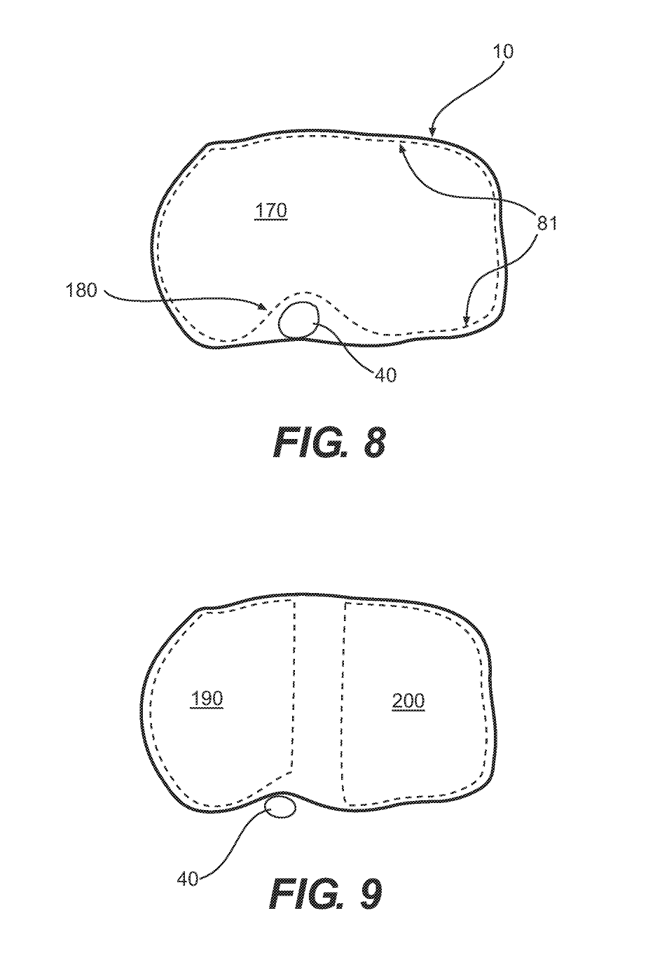

FIG. 8 depicts an alternative embodiment of a tibial tray 170 for use with a PCL retaining implant system. In this embodiment, a perimeter 81 of the tibial tray 170 substantially matches the perimeter 10 of the resected tibia, except for a notched section 180 which is desirably located proximate the PCL 40. In this embodiment, the ACL has not been retained, for any variety of reasons, but the PCL and related supporting structure (e.g., underlying bony anatomy) can be accommodated by the implant.

FIG. 9 depicts an alternative embodiment of another set of tibial tray components 190 and 200, which have been selected and/or designed to accommodate the unique placement of a patient's PCL 40. In this embodiment, the PCL 40 is displaced posteriorly relative to the tibial surface, which facilitates the design and placement of the components 190 and 200 while allowing the retention of the PCL.

Implant design and modeling also can be used to achieve ligament sparing, for example, with regard to the PCL and/or the ACL. An imaging test can be utilized to identify, for example, the origin and/or the insertion of the PCL and the ACL on the femur and tibia. The origin and the insertion can be identified by visualizing, for example, the ligaments directly, as is possible with MRI or spiral CT arthrography, or by visualizing bony landmarks known to be the origin or insertion of the ligament, such as, for example, the medial and lateral tibial spines.

An implant system can then be selected or designed based on the image data so that, for example, the femoral component preserves the ACL and/or PCL origin, and the tibial component preserves the ACL and/or PCL attachment. The implant can be selected or designed so that bone cuts adjacent to the ACL or PCL attachment or origin do not weaken the bone to induce a potential fracture.

For ACL preservation, the implant can include a notch or other opening that can be selected or designed and placed using the image data. Alternatively, the implant can have an anterior bridge component. The width of the anterior bridge in A/P dimension, its thickness in the superoinferior dimension or its length in mediolateral dimension can be selected and/or designed using the imaging data and, specifically, the known insertion of the ACL and/or PCL.

As can be seen in FIGS. 8 and 9, the posterior margin of an implant component, e.g., a polyethylene- or metal-backed tray with polyethylene inserts, can be selected and/or designed using the imaging data or shapes derived from the imaging data so that the implant component will not interfere with and stay clear of the PCL. This can be achieved, for example, by including concavities, notches or other features in the perimeter of the implant and/or insert(s) that are specifically designed or selected or adapted to avoid the ligament insertion. Similar design considerations can be utilized in conjunction with other relevant or pertinent connective tissue structures.

Any implant component can be selected and/or adapted in shape so that it stays clear of important ligament structures. Imaging data can help identify or derive shape or location information on such ligamentous structures. For example, the lateral femoral condyle of a unicompartmental, bicompartmental or total knee system can include a concavity or divot to avoid the popliteus tendon. Imaging data can be used to design a tibial component (all polyethylene or other plastic material or metal backed) that avoids the attachment of the anterior and/or posterior cruciate ligaments; specifically, the contour of the implant can be shaped so that it will stay clear of these ligamentous structures. A safety margin of, e.g., about 2 mm or about 3 mm or about 5 mm or about 7 mm or about 10 mm, can be applied to the design of the edge of the component, which can allow the surgeon more intraoperative flexibility.

Similar features can be incorporated into other joints, including in a shoulder, where the glenoid component can include a shape or concavity or divot to avoid a subscapularis tendon or a biceps tendon. Similarly, in a hip the femoral component can be selected or designed to avoid an iliopsoas or adductor tendons.

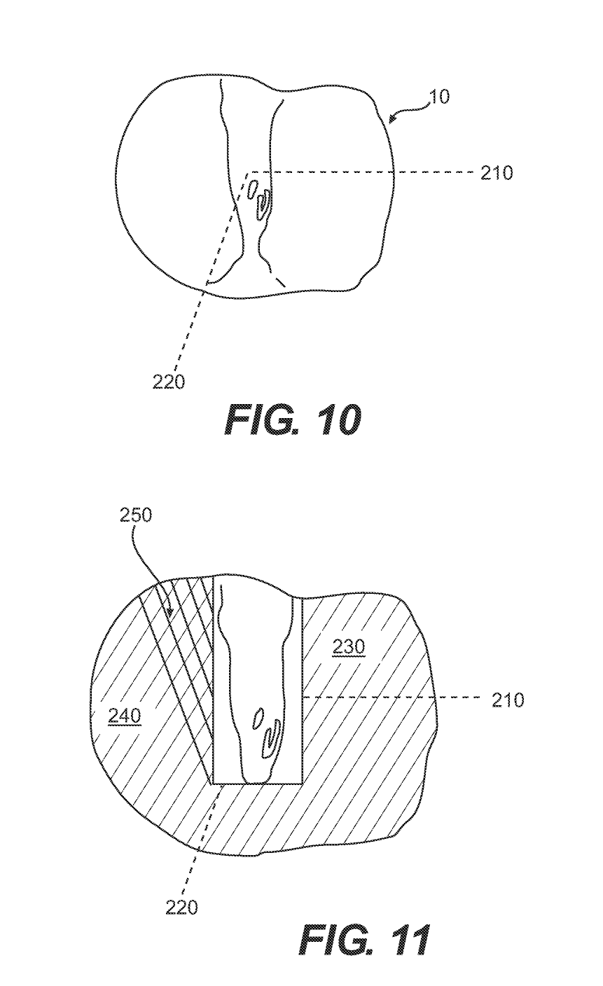

FIG. 10 depicts a top plan view of an unresected surface of a tibia 10. In a typical less-invasive and/or minimally-invasive surgical procedure, a surgical window through the skin and overlying tissues (for access to the relevant femoral and tibial structures of the knee) may extend clockwise from approximately three o'clock (line 210) to no greater than approximately 7 o'clock (line 220). This window will generally extend from the medial collateral ligament (see 30, FIG. 6) to the patellar tendon (see 25, FIG. 1). In many instances, the window may be “stretched” slightly towards one side or the other by distracting the relevant ligament/tendon structure, while allowing the opposite side of the window to relax to some limited degree. In various embodiments, the access window will desirably allow surgical access to relevant knee structures with minimal tissue disruption.

While less invasive and/or minimally invasive access procedures may be preferred, a significant limitation in using some such approaches, as compared to open procedures, can be that a medial surgical window significantly limits direct access to the lateral aspect of the tibia. As best seen in FIG. 11, while the surgeon can easily visualize the entire medial compartment 230 of the tibia and access such with surgical tools, a much larger percentage of the lateral compartment 240 is at least partially masked by overlying tissues and/or other intermediate structures. Moreover, at least a portion of the lateral compartment directly adjacent the posterior aspect of the central region cannot be visualized or directly accessed without additional retraction of the patellar tendon, which may be impossible or undesirable for many reasons. In FIG. 11, one exemplary region difficult to visualize and/or access is identified by the cross-hatched section 250.

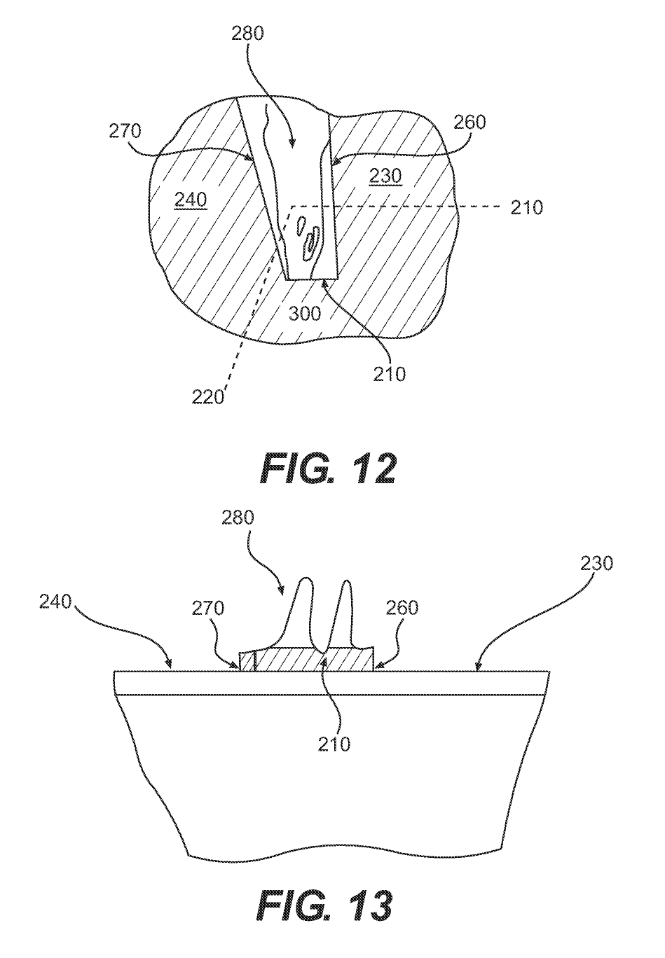

Various embodiments and procedures described herein include features that can desirably accommodate and/or account for the visualization and/or access difficulties previously described in connection with some less invasive and/or minimally invasive access windows. FIGS. 12 and 13 depict one such procedure, in which the medial and lateral compartments 230 and 240 have been prepared with one or more canted or angled substantially vertical walls 260 and 270 which border a central region 280. The central region further includes a substantially vertical anterior wall 210, which borders an anterior bridge accommodating surface 300 formed on the tibial surface. Desirably, in various embodiments the central region 280 will maintain a minimum width and comprise sufficient material to maintain its desired structural integrity, as well as provide sufficient anchoring material for the ACL and/or PCL.

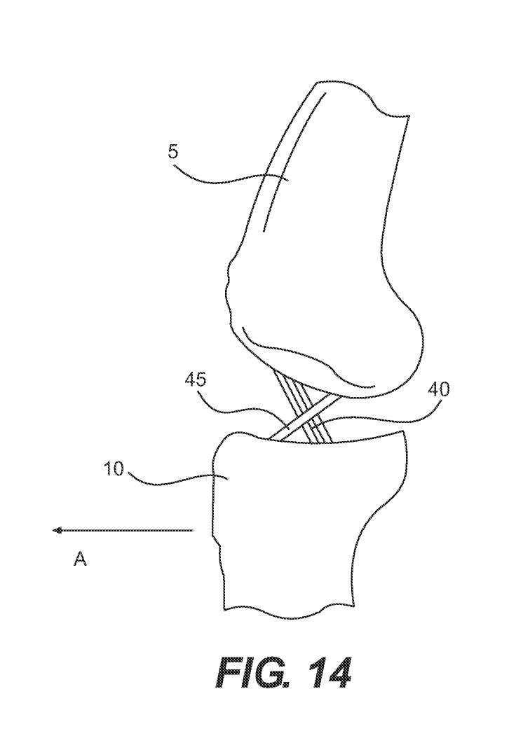

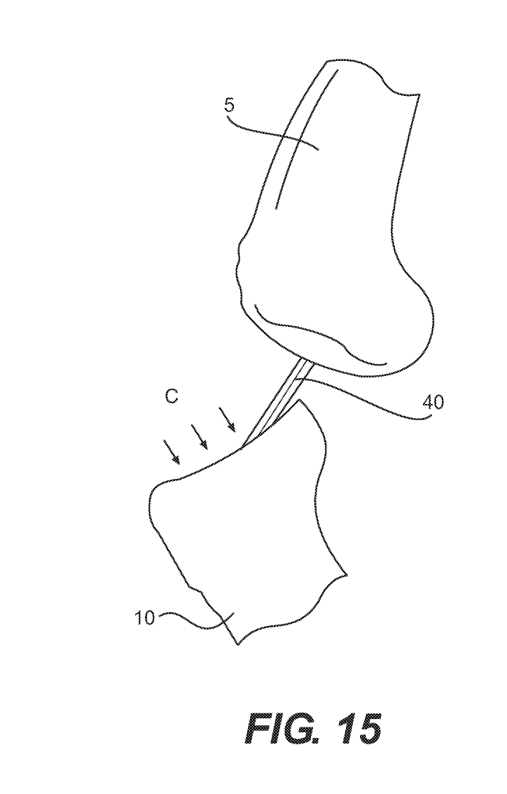

Various embodiments described herein facilitate the retention of both the PCL and ACL, which can significantly impact the surgical procedure in a variety of ways. For example, where an ACL is sacrificed, damaged or is otherwise deemed unnecessary, the removal of such structure often improves the ability of the surgeon to access the tibial and/or femoral surfaces. For example, FIG. 14 depicts a schematic side view of a knee joint, wherein the femur 5 and tibia 10 are connected together via the flexible structures of the ACL 45 and PCL 40. While a healthy ACL and PCL cooperate to allow the femur 5 to rotate relative to the tibia 10 (in a known manner and relationship), the ligaments also further cooperate to limit relative motion between the tibia and femur in an anterior/posterior direction. When the ACL 45 is severed or otherwise released, the tibia can be advanced some distance anterior relative to the femur (in direction “A” indicated in FIG. 14), which allows the surgeon to dislocated the knee to some degree and gain access to the upper surface of the tibia from a more cephalad orientation (direction “C” as indicated FIG. 15). In a similar manner, severing or release of the PCL can facilitate some degree of advancement of the femur relative to the tibia.

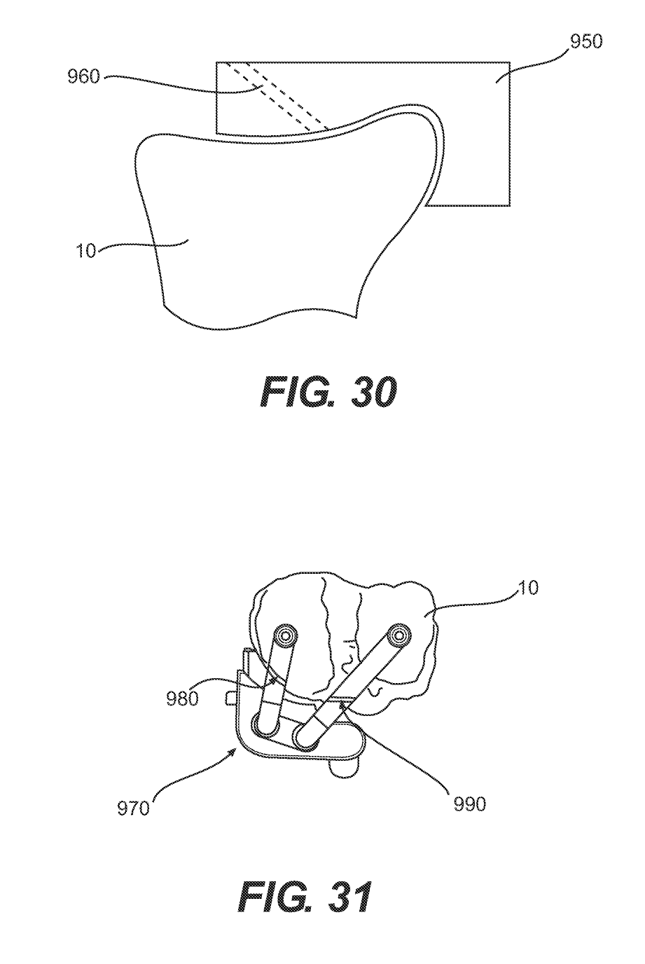

In various embodiments described herein, the release of the ACL can facilitate the use of guide tools, jigs and/or surgical tools on various exposed surfaces of the tibia. For example, various jigs and procedures described herein, such as, for example, the jigs and surgical steps described in conjunction with FIGS. 29 and 30, can be more easily accommodated and performed when the tibia has been advanced relative to the femur. If desired, the various procedures and systems described herein can further include the employment of ligament repair and/or replacement procedures which can restore various tissue structures, including the employment of natural or artificial ACL and/or PCL structures, after the various joint replacement and/or resurfacing procedures described herein have been accomplished. In conjunction with such procedures, the tibial tray can, optionally, incorporate a posterior bridge (either in place of or in addition to the anterior bridge portion), with the tibial tray implanted prior to repair and/or replacement of the ACL and/or PCL structures. In various embodiments, such a system can include e a tibial tray implant including anterior and posterior bridge portions that completely encompasses a centrally-located remainder portion of the tibial surface (with such tibial anatomy capable of including attachment locations for a replacement ACL and/or PCL).

Where both the ACL and PCL have been retained, however, a surgeon's direct access to the upper surface of the tibia may be limited to the anterior face of the tibia with some limited access space between the articulating surfaces of the femur and tibia. Moreover, where such access is accomplished via a less-invasive and/or minimally invasive approach, the constraints on direct access can increase even further. Accordingly, various embodiments described herein facilitate the surgical repair and/or replacement of tibial and/or femoral articulating surfaces and associated structures via a less-invasive and/or minimally invasive approach. In addition, various embodiments described herein can be utilized with equal effectiveness in open surgical procedures where the ACL and/or PCL have been retained.

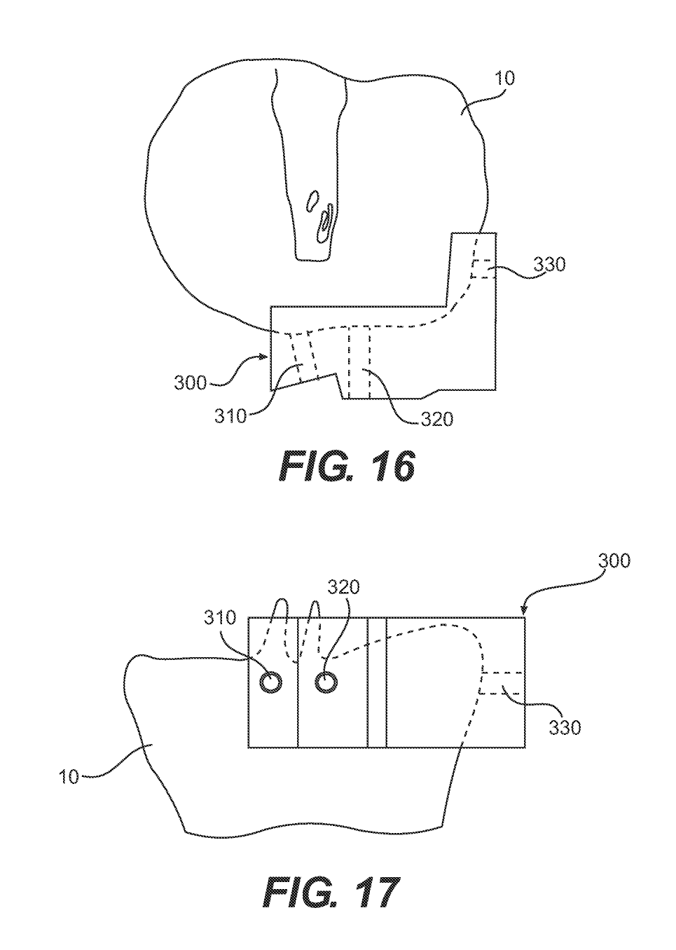

FIGS. 16 and 17 depict top and front views, respectively, of one embodiment of a tibial jig 300 for preparing a tibia 10 for receiving a ligament sparing tibial tray. The jig includes an inner surface (not shown) that substantially conforms to a natural surface of the tibia that is exposed and accessible through the surgical incision. Various aspects of the jig, as well as the implant components described herein, can be manufactured to incorporate one or more patient-specific and/or patient-engineered surfaces using noninvasive imaging data of the patient's anatomy, as described in U.S. patent application Ser. No. 13/397,457 to Bojarski et al, filed Feb. 15, 2012, which is entitled “Patient-Adapted and Improved Articular Implants, Design and Related Guide Tools,” and published as US Patent Publication No. 2012-0209394, the entire disclosure of which is incorporated herein by reference.

Desirably, the conforming surface of the jig will mate with the substantially matching surface of the tibial anatomy, positioning the jig in a known position and orientation relative to the tibial surfaces. A series of guide channels and/or slots, such as 310, 320 and 330, can be provided in the jig 300. For example, as depicted in FIGS. 16 and 17, guide channels 310, 320 and 330 are drill channels for guiding a drill along a known trajectory into the tibia. If desired, the thickness of the jig 300 along the longitudinal axis of the respective drill channels can be modified and/or tailored to act as drill “stops,” thereby preventing the drill from exiting the posterior surface of the tibia (after passing into and through the drill channel and bone) and potentially damaging surrounding soft tissues. Once all three drill channels have been utilized for drilling, one or more pins (not shown) can be inserted into the bone and/or the jig can be removed.

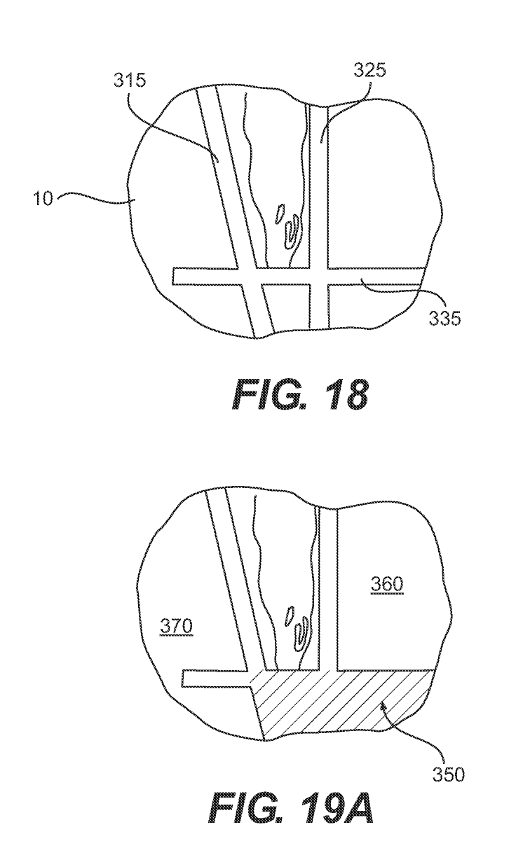

FIG. 18 depicts the tibia after removal of the jig, with three drill channels 315, 325 and 335 formed therein. The anterior/posterior facing channels 315 and 325 can be parallel, or non-parallel, as depicted in FIG. 18. A lateral channel 335 also extends across the tibia, and in the depicted embodiment the lateral channel 335 intersects with the substantially A/P channels 315 and 325. If desired, the channels need not intersect, and various combinations of channels can be utilized.

If desired, one or more of the channels 315, 325 and/or 335 can be utilized as reference and/or guide points for further procedural steps. For example, a second jig can employ one or more guide pins that fit into one or more of the corresponding channels 315, 325 and/or 335, previously formed in the tibia, as guide points or other alignment features. The guide pin locations can then be utilized to align and orient the second jig. The second jig, in turn, can incorporate one or more guide channels and/or slots for guiding surgical tools utilized to continue preparing the tibial surface for one or more tibial tray implants. In various embodiments, the creation of two anterior (or other orientation) channels (as previously described) in a relatively parallel orientation may further facilitate the use of additional jigs with corresponding guide pins (for placement in the anterior channels), which can be slid on and off the pins without requiring removal of the pins from the bone channels. Various jig designs can include virtually any number of guide surfaces and/or drill channel guides, including 10, 9, 8, 7, 6, 5, 4, 3, 2 or 1 guide surfaces, slots and/or channels per individual jig or group of jigs.

In various embodiments, the one or more drill channels can be positioned and/or oriented to desirably mark a mesial (or other) boundary for intended further surgical cuts and/or serve as location(s) and/or reference feature(s) for intended implant placement. For example, a jig or other alignment guide can used to place two parallel (or other oriented) channels on the medial and lateral sides of the central region, and then these channels (or pins or other features occupying these channels) can be further used to orient a wide variety of surgical cutting, drilling, rongeuring, rasping and/or other tools. Moreover, in various embodiments the drill channels themselves can form a portion of the “prepared tibial surfaces” for receiving the implant, with various surgically created surfaces extending into and/or out of the drill channels, and with at least a portion of the tibial tray implant extending into one or more of the location(s) where the drill channels were initially formed.

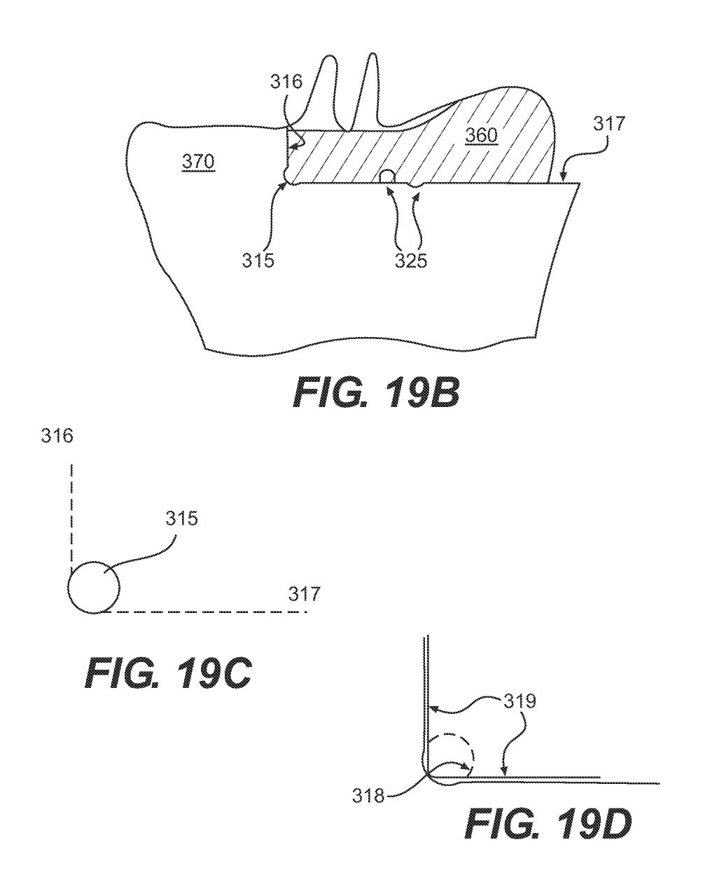

FIG. 19A depicts the tibia of FIG. 18, with various combinations of additional surgical cutting, drilling, grinding and/or rongeuring tools employed to remove various sections of the tibia in preparation for the tibial tray implant. In this embodiment, a cutting tool can initially be employed to cut bone in a substantially vertical orientation along a substantially anterior/posterior path using the lateral side A/P channel 315 as a guide. The cutting tool can then be employed to cut bone in a substantially vertical orientation along a substantially lateral path using the lateral channel 335 as a guide. The cutting tool can then be employed to cut bone in a substantially horizontal orientation using the lateral channel 335 as a guide. When completed, an anterior portion 350 of the tibial surface can be removed (see FIGS. 19A and 19B). In alternative embodiments, cutting steps may be performed in differing order and/or additional jigs may be employed to guide the various cutting tools along the desired paths described herein.

FIG. 19B depicts a frontal perspective view of the tibia of FIG. 19A, with portions of the drill channels 325 and 315 forming sections of the prepared tibial surface. As can be seen in FIG. 19C, the cut planes 316 and 317, that are followed using surgical cutting tools, will extend into and/or out of the drill channel 315. FIG. 19D depicts implant walls 319 positioned adjacent the cut planes, with at least a portion of the implant extending into the originally-formed drill channel (the original boundary of which is indicated by dotted line 318).

In many surgical procedures, drill channels formed in bone using alignment jigs and/or other guide tools can often be more accurate in their placement and orientation than are cut planes created using saws and/or other cutting tools. This can often be due to flexure/deformation of the cutting elements and/or the effects of harder versus softer bone, which can often skew or deflect the sawing or cutting tools to some degree. In various embodiments, therefore, it may be desirable to form one or more drill channels at various boundaries of cutting planes (e.g., corners, with the drill channels being used as guide points, starting points and/or ending points for planar cutting tools and/or using the drill channels themselves to form some or all of the prepared bone surface.

In subsequent steps, the medial portion 360 and lateral portion 370 of the tibial surface can be removed (if desired, using similar cutting tools and techniques). In some embodiments, the retention of the ACL and PCL, and the associated tension within the knee joint, substantially limits surgical access to the top of the tiba. In such cases, the use of cutting tools and paths advanced along the anterior and lateral faces of the tibia (substantially horizontally and limited from the vertical or cephalad direction) allows for removal of relevant structures and preparation for the tibial tray implant. If desired, various other guide tool arrangements, including open-faced guide tools allowing router or rongeur access to the face of the tibia to shape desired surface planes and/or structures, can be utilized.

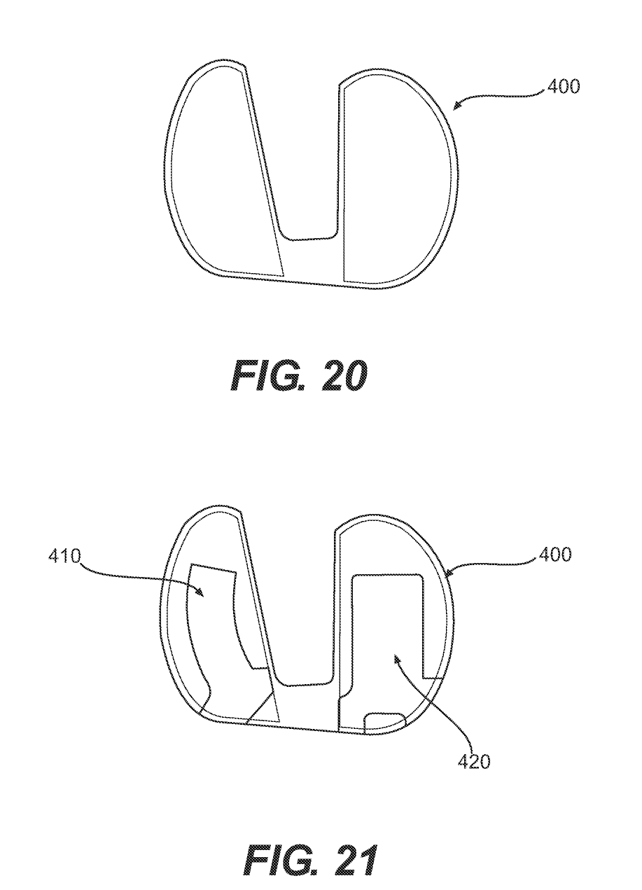

FIG. 20 depicts one embodiment of a tibial tray 400 for use in an ACL/PCL retention procedure. Optionally, the outer profile of the tray has been selected, designed and/or adapted to substantially match or otherwise accommodate the outer profile of the tibial surface (e.g., it, optionally, does not overhang the tibial surface at locations adjacent to soft tissue structures such as the MCL, the LCL and/or the patellar tendon, etc.). In a similar manner, the inner perimeter or “notch” of the tray 400 that accommodates the remaining anatomical structures of the tibia has, optionally, been selected, designed and/or adapted to accommodate such remaining structures, and/or such structures have been modified to match or be accommodated by the notch. Once the tibial surface has been properly prepared, the tray 400 can be positioned on the tibia and secured using standard attachment mechanisms, including posts, stems, screws, bone cement and/or the like.

In various alternative embodiments, the tibial tray and/or insert(s) can be selected (e.g., preoperatively or intraoperatively) from a collection or library of implants for a particular patient (e.g., to best-match the perimeter of the patient's cut tibial surface) and implanted without further alteration to the perimeter profile. However, in certain embodiments, different tibial tray and/or insert perimeter profiles can serve as blanks. For example, a tibial tray and/or insert profile can be selected preoperatively from a library (e.g., an actual or virtual library) for a particular patient to best-match the perimeter of the patient's cut tibial surface. Then, the selected implant perimeter can be designed or further altered based on patient-specific data, for example, to substantially match the perimeter of the patient's cut tibial surface.

If desired, various features of a tibial implant component can be designed or altered based on patient-specific data. For example, the tibial implant component design or alterations can be made to maximize coverage and extend to cortical margins; maximize medial compartment coverage; minimize overhang from the medial compartment; avoid internal rotation of tibial components to avoid patellar dislocation; and avoid excessive external rotation to avoid overhang laterally and impingement on the popliteus tendon. The amount of “perimeter matching” of the tray to the tibia may vary widely, ranging from an extremely “organic” design that may substantially match the tibial perimeter in every detail, to a more smoothed or regular geometric shape design that approximates and covers some portion of, but not all, of the cortical margin of the tibia. Tray designs may also include perimeter designs that “filter” the exact contours of the tibial perimeter, creating a tray perimeter that grossly, but not exactly, follows the tibial perimeter. Similar design consideration can be utilized in designing, selecting and/or shaping the notch of the implant.

FIG. 21 depicts one embodiment of a tibial tray 400 including a pair of attachment or locking mechanisms 410 and 420 for securing polyethylene inserts to the upper surface of the tray 400. In this embodiment, the medial mechanism 420 can be configured to accept a tibial insert having a straight engagement portion (not shown) while the lateral mechanism 410 is configured to accept a tibial insert having a curved engagement portion (not shown). This arrangement can be especially well suited for placement of inserts through a less-invasive or minimally-invasive surgery. While the medial insert can be advanced from anterior to posterior through the incision, the lateral insert would typically be inserted at an angle from the medial side, and then advanced and/or rotated into engagement with the locking mechanism 410. In various alternative embodiments, the locking mechanisms could include straight and/or curved mechanisms on either or both of the medial and lateral sides.

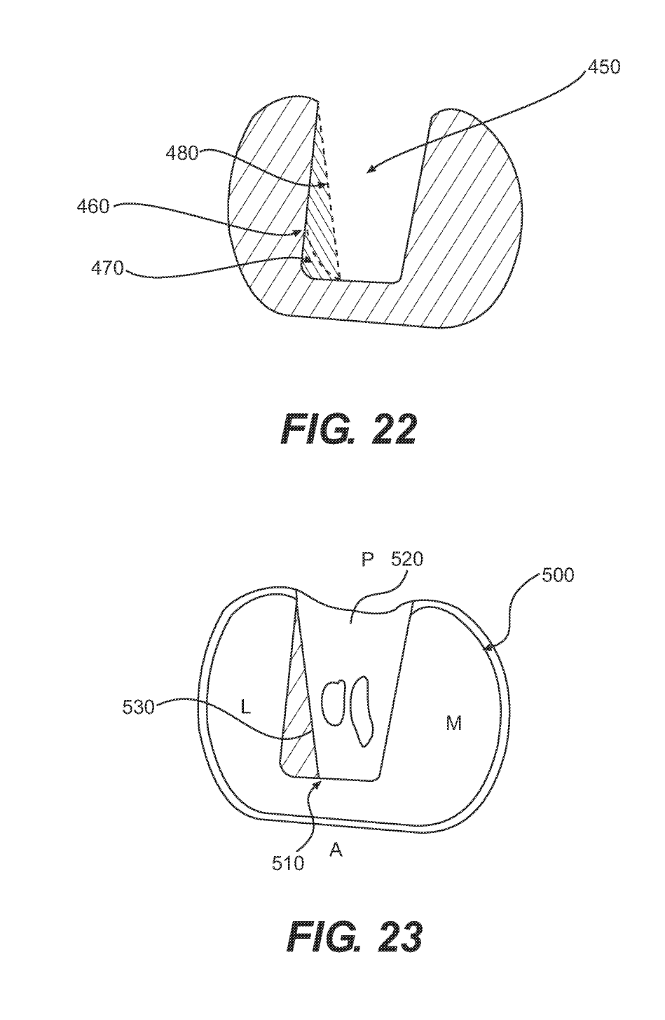

FIG. 22 depicts a diagram of a tibial surface prepared in accordance with various teachings disclosed herein. In this embodiment, the central region 450 includes a resected portion 460 that can be removed to facilitate access to the lateral side of the tibial surface. If desired, the resection can include a curved surface 470, a straight surface 480 or various combinations thereof, as desired by the physician. In addition, the edges of the central region 450 can be smoothed or otherwise shaped, as desired.

FIG. 23 depicts one alternative embodiment of a tibial tray having a perimeter 500 and incorporating a notched section 510 to accommodate a remaining natural section 520 of the tibial surface. In this embodiment, additional material has been removed from a lateral side of the central region, but the tray 500 does not completely fill this region, leaving a void 530. In such a case, the tray 500 can still be utilized, although the physician may, optionally, choose to fill the void 530 with various materials, which could include bone cement, bone graft and/or a plug or insert (not shown).

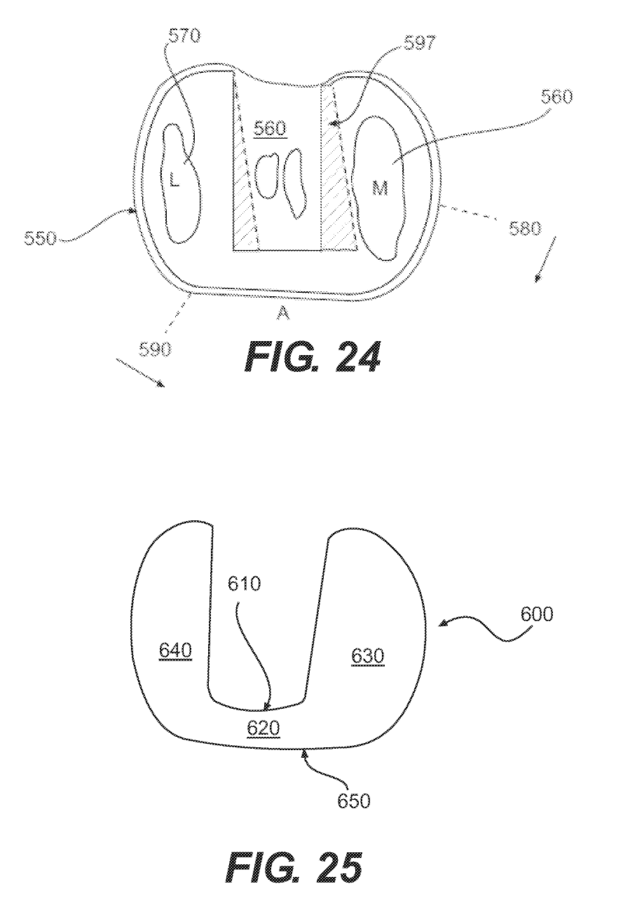

FIG. 24 depicts a top plan view of a tibial tray having a perimeter 550 and exemplary medial and lateral articulating surfaces 560 and 570. An exemplary less-invasive surgical “window” (clockwise from 580 to 590) for accessing the knee surfaces. The window extends from the medial side 580 to 590, slightly lateral of the anterior midline of the knee. Also depicted are two optional alternative notch side walls (optional lateral notch wall 595 and optional medial notch wall 597). Depending upon the surgical access as well as the tray design, the notch can comprise a plurality of dimensions, including those depicted by dashed line 597. If desired, the notch may be sized and/or configured to accommodate a plurality of shapes and/or sizes for the central region 560, with additional space between the inner surfaces of the notch and the central region being occupied by spacers, bone graft, bone cement or other materials and/or substantially left as one or more voids. Where the notch is intentionally “oversized” in various dimensions relative to the central region, such a configuration may facilitate various degrees of rotation of the tray relative to the cut tibial surface, allowing the physician some degree of flexibility during implantation of the device.

FIG. 25 depicts one alternative design for an embodiment of a tibial tray 600, including a rounded or curved inner surface of the notch region 610. In this embodiment, the use of curved and/or chamfered inner surfaces can significantly increase the strength of the anterior bridge 620 which connects the medial tray portion 630 to the lateral tray portion 640 (e.g., by reducing potential regions and/or shapes susceptible to stress concentrations, especially where the material may be particularly notch-sensitive). If desired, the anterior perimeter of the tray 600 can similarly include one or more curved outer surfaces 650, although virtually any surface shape and/or features accommodated by the prepared tibial surface could be employed.

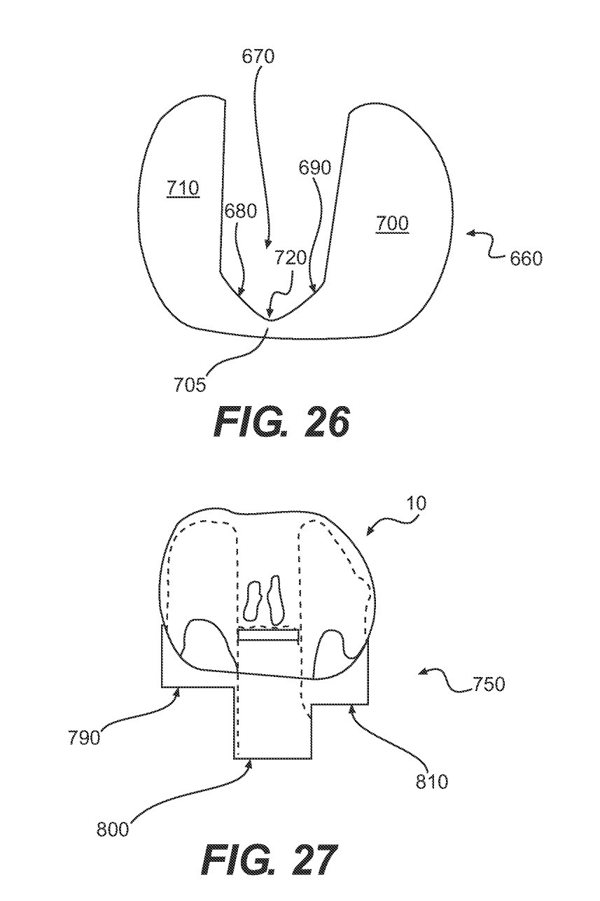

FIG. 26 depicts another alternative design for an embodiment of a tibial tray 660, including one more flattened and/or angled inner surfaces of the notch region 670. In this embodiment, the use of angled and/or flattened surfaces such as 680 and 690 allow for greater A/P thickness of the anterior bridge 705 proximate the locations where the bridge contacts the medial tray portion 700 and the lateral tray portion 710, but allows for reduced thickness at the centerline 720 of the bridge 705. This arrangement can allow for retention of additional natural anatomical structures adjacent the notch, and the use of flattened surfaces can potentially facilitate the cutting and/or shaping of the corresponding natural anatomy. If desired, various combinations of angled, flattened, curved, chamfered and/or tapered inner surfaces (as well as other lateral, medial, inferior, superior, internal and/or external surfaces of the tibial tray and/or its components) are contemplated herein.

In addition to optimizing bone preservation, avoiding various connective or other tissues and/or other surgical considerations, another factor in determining the depth, number, and/or orientation of resection cuts and/or implant component bone cuts is desired implant thickness. One or more minimum implant thicknesses in varying orientations can be included as part of the resection cut and/or bone cut design (as well as part of implant design) to ensure a threshold strength for the implant in the face of the stresses and forces associated with joint motion, such as standing, walking, and running. In various embodiments, a finite element analysis (FEA) assessment for various implant components can be conducted for various sizes and with various bone cut numbers and orientations. The maximum principal stress observed in FEA analysis can be used to establish an acceptable minimum implant thickness for an implant component having a particular size and, optionally, for a particular patient (e.g., having a particular weight, age, activity level, etc). Before, during, and/or after establishing a minimum implant component thickness, the optimum depth of resection cuts and optimum number and orientation of resection cuts and bone cuts, for example, for maximum bone preservation, can be designed.

In certain embodiments, an implant component design or selection can depend, at least in part, on a threshold minimum implant component thickness as well as other strength and/or durability considerations driven by, for example, FEA assessment. In turn, the threshold minimum implant component thickness or other dimensions can depend, at least in part, on patient-specific data, such as condylar width, central tibial region width, tibial dimensions and/or the patient's specific weight. In this way, the threshold implant thickness, and/or any implant component feature, can be adapted to a particular patient based on a combination of patient-specific geometric data and on patient-specific anthropometric data. This approach can apply to any implant component feature for any joint, for example, the knee, the hip, or the shoulder.

If desired, computerized modeling of the implant, the anatomy and/or combinations thereof can be utilized to virtually determine a resection cut strategy for the patient's femur and/or tibia that provides minimal bone loss, optionally, while also meeting other user-defined parameters, such as, for example, maintaining a minimum implant thickness, using certain resection cuts to help correct the patient's misalignment, removing diseased or undesired portions of the patient's bone or anatomy, and/or other parameters. This general step can include one or more of the steps of (i) simulating resection cuts on one or both articular sides (e.g., on the femur and/or tibia), (ii) applying optimized cuts across one or both articular sides, (iii) allowing for non-co-planar and/or non-parallel femoral resection cuts (e.g., on medial and lateral corresponding portions of the femur) and, optionally, non-co-planar and/or non-parallel tibial resection cuts (e.g., on medial and lateral corresponding portions of the tibia), and (iv) maintaining and/or determining minimal material thickness. The minimal material thickness for the implant selection and/or design can be an established threshold, for example, as previously determined by a finite element analysis (“FEA”) of the implant's standard characteristics and features (or analysis of individual portions of the implant such as, for example, the anterior bridge or other regions). Alternatively, the minimal material thickness can be determined for the specific implant, for example, as determined by an FEA of the implant's standard and patient-specific characteristics and features. If desired, FEA and/or other load-bearing/modeling analysis may be used to further optimize or otherwise modify the individual implant design, such as where the implant is under or over-engineered than required to accommodate the patient's biomechanical needs, or is otherwise undesirable in one or more aspects relative to such analysis. In such a case, the implant design may be further modified and/or redesigned to more accurately accommodate the patient's needs, which may have the side effect of increasing/reducing implant characteristics (e.g., size, shape or thickness in global and/or localized areas of the implant) or otherwise modifying one or more of the various design “constraints” or limitations currently accommodated by the present design features of the implant. If desired, this step can also assist in identifying for a surgeon the bone resection design to perform in the surgical theater and it also identifies the design of the bone-facing surface(s) of the implant components, which substantially negatively-match the patient's resected bone surfaces, at least in part.

By optimizing implant shape in this manner, it is possible to establish normal or near normal kinematics. Moreover, it is possible to avoid implant related complications, including but not limited to implant complications such as anterior notching, notch impingement, posterior femoral component impingement in high flexion, and other complications associated with existing implant designs. Similar implant complications can be avoided for tibial components as well. For example, certain designs of the femoral components of traditional knee implants have attempted to address limitations associated with traditional knee implants in high flexion by altering the thickness of the distal and/or posterior condyles of the femoral implant component or by altering the height of the posterior condyles of the femoral implant component. Since such traditional implants follow a one-size-fits-all approach, they are limited to altering only one or two aspects of an implant design. However, with the design approaches described herein, various features of an implant component can be designed for an individual to address multiple issues, including issues associated with high flexion motion. For example, designs as described herein can alter an implant component's bone-facing surface (for example, number, angle, and orientation of bone cuts), joint-facing surface (for example, surface contour and curvatures) and other features (for example, implant height, width, and other features) to address issues with high flexion together with other issues.

Biomotion models for a particular patient can be supplemented with patient-specific finite element modeling or other biomechanical models known in the art. Resultant forces in the knee joint can be calculated for each component for each specific patient. The implant can be engineered to the patient's load and force demands. For instance, a 1251b. patient may not need a tibial plateau as thick as a patient with 280 lbs. Similarly, the polyethylene can be adjusted in shape, thickness and material properties for each patient. For example, a 3 mm polyethylene insert can be used in a light patient with low force and a heavier or more active patient may need an 8 mm polymer insert or similar device.

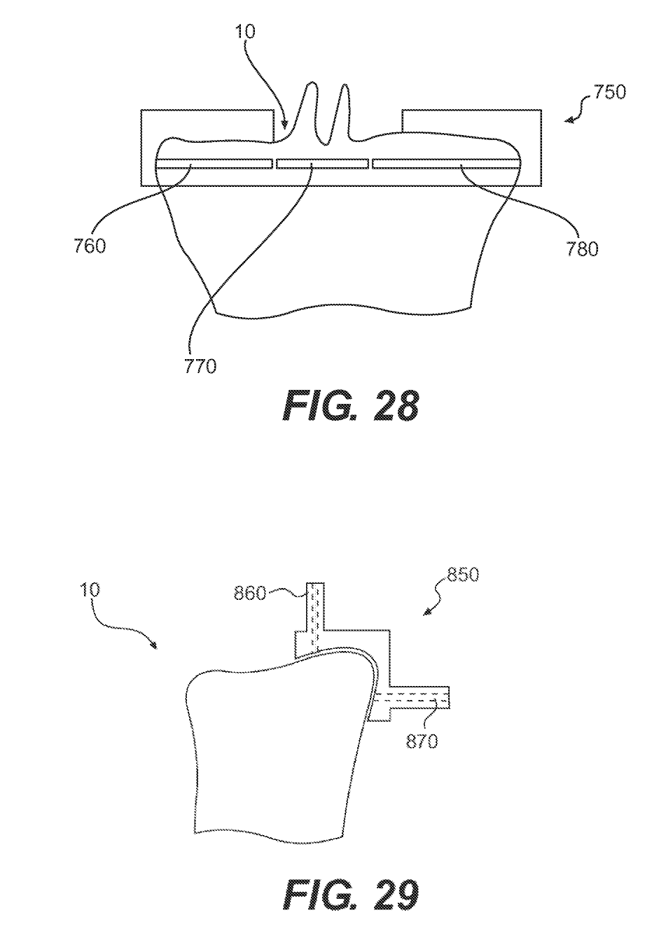

FIGS. 27 and 28 depict top plan and side views of an alternative embodiment of a surgical cut or guide tool 750 for use in preparing portions of the surface of the tibial bone for a tibial tray implant. In various embodiments, and as previously described in connection with the tool of FIGS. 16 and 17, at least one inner surface of the tool 750 includes one or more surface features that match, conform to or otherwise accommodate patient-specific features of the tibial bone, facilitating placement of the tool in a known position and/or orientation relative to the tibia 10. As best seen in FIG. 28, a series of cutting guides or slots 760, 770 and 780 are formed in the anterior face of the tool, and these slots extend through the posterior side of the tool. These slots desirably can be used to guide one or more cutting tools (not shown) along a desired trajectory, thereby creating, in this example, a series of lateral cuts across the upper surface of the tibia. As can best be seen in FIG. 27, the tool 750 further includes one or more patient-specific anterior surfaces or “stops” 790, 800 and 810, which can comprise differing thicknesses of the tool 750 in an anterior direction. Such stops can be configured to limit the penetration depth of the cutting instrument into the tibial bone, optionally, preventing the tool from exiting the posterior side of the tibial bone and possibly damaging posterior tissues, which may be difficult to visualize as the surgeon cuts. Moreover, the central stop 800 optionally prevents the cutting tool from advancing across the entire width of the tibia, thereby facilitating creation and retention of a raised central region of the tiba, as previously described.

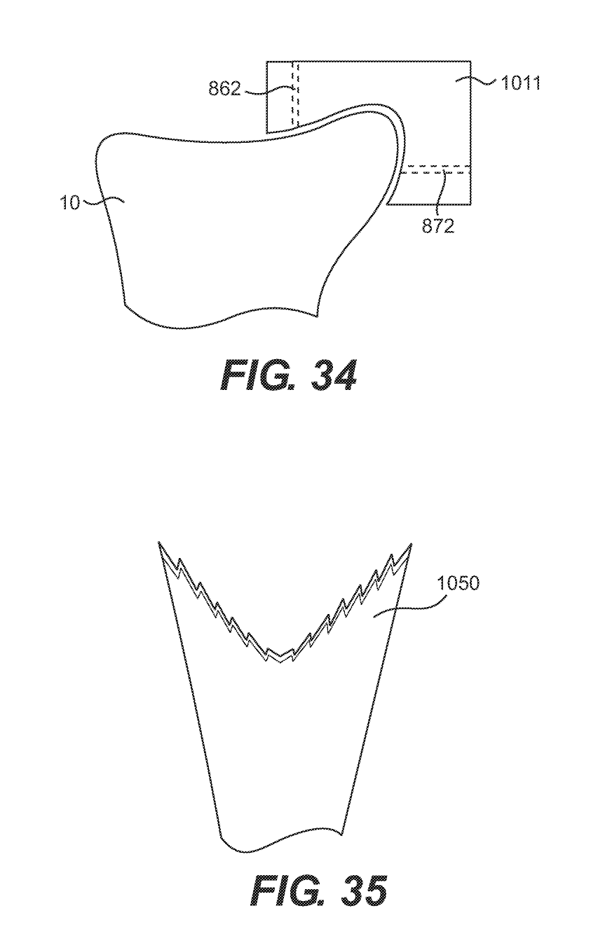

FIG. 29 depicts a side plan view of an alternate embodiment of a tibial guide tool or jig 850 for use in preparing the surface of a tibia 10 for receiving a tibial tray implant. In this embodiment, at least a portion of the jig 850 extends over the superior surface of the tibia, and a remainder of the jig extends around a portion of the periphery of the tiba 10 (the periphery could include an anterior, posterior, medial and/or lateral peripheral face, or various combinations thereof). The jig 850 can include at least first and second alignment orifices 860 and 870 extending there through. In this embodiment, the alignment orifices are nonparallel, although virtually any combination and/or position/orientation relationship between the orifices are contemplated herein. In this specific embodiment, the first alignment orifice 860 is vertically oriented into the tibia, and the second alignment orifice 870 is horizontally oriented into to the tibia. The alignment orifices are utilized to create defined drill and/or cut channels in the tibia. If desired, the drill channels can be utilized to contain one or more alignment pins (not shown) that can be used to align subsequent surgical instruments and/or jig for additional surgical steps. One alternative embodiment of this jig is depicted in FIG. 34. If desired, the individual alignment orifices described herein can comprise parallel sets of spaced alignment orifices, with each orifice set being non-coplanar to other orifice sets on the same jig.

FIG. 30 depicts a side plan view of another alternate embodiment of a tibial guide tool or jig 950 for use in preparing the surface of a tibia 10 for receiving a tibial tray implant. In this embodiment, the jig includes an inner surface that conforms to some degree to the natural tibial structures, and also includes at least one angled alignment orifice 960 extending through the body of the jig.

FIG. 31 depicts a jig 970 having external indicia 980 and 990 that substantially match or indicate one or more features of the targeted anatomy, and/or identify features of interest for the surgeon's reference. In this jig 970, a series of indentations 980 and 990 are formed on support arms such that, when the jig is properly aligned relative to a targeted anatomy (in this instance, a tibia 10), the indentations align with a perimeter edge of the body as seen from a cephalad direction of the tibia. These features permit the surgeon to verify proper alignment of the jig 970 prior to employing cutting tools or other surgical instruments.

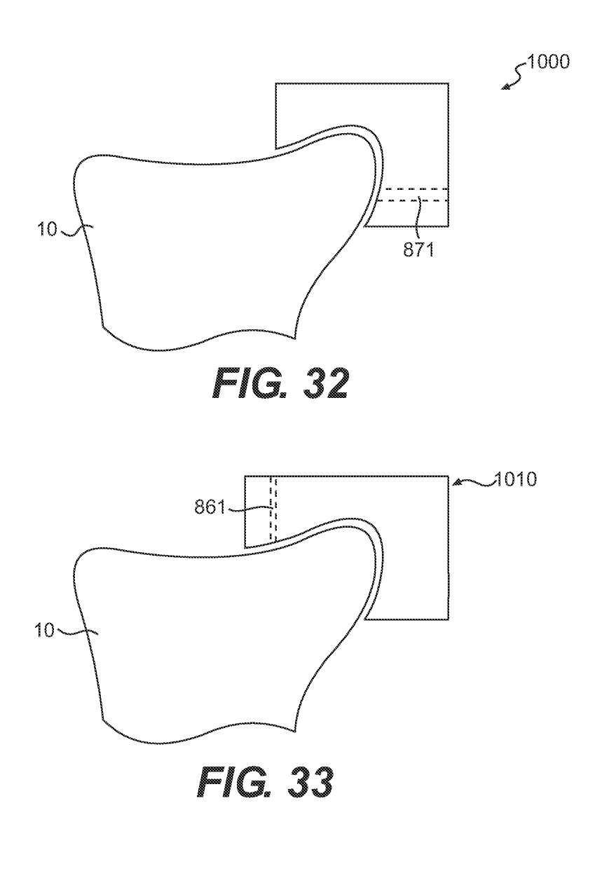

FIGS. 32 and 33 depicts a set of jigs 1000 and 1010 for use in creating a plurality of cut planes and/or other surgical objectives (e.g., drill holes, etc.), with each jig 1000 and 1010 incorporating a substantially matching and/or conforming inner surface for engaging a portion of the tibial surface. The various inner surfaces need not engage the same section of the tibia, and it is contemplated that the inner surface for a subsequent jig could incorporate and/or accommodate surgical alterations performed at earlier stages of the procedure. For example, jig 1010 could include surface features that correspond to drill holes and/or cut planes created by a surgeon using jig 1000.

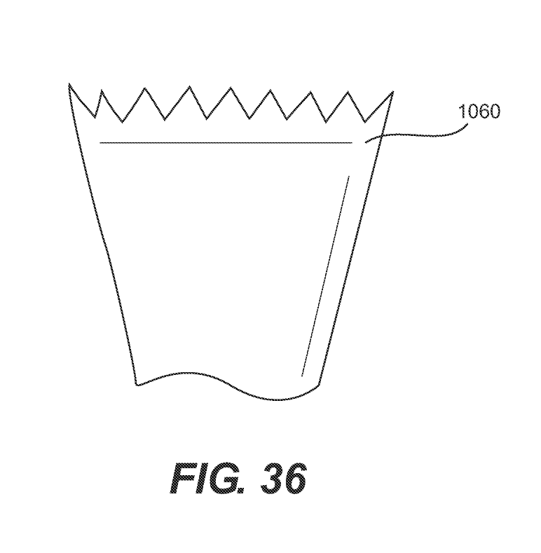

FIGS. 35 and 36 depict alternative embodiments of surgical cutting instruments, such as saw blades 1050 and 1060, which may be particularly useful with various embodiments described herein. Such blades can be employed in conjunction with reciprocating and/or vibratory power sources, which can advance/withdraw, laterally slide and/or rotate the various cutting surfaces in a desired direction and/or orientation to accomplish a cutting and/or drill operation. For example, the cutting instrument 1060 shown in FIG. 36 could be laterally vibrated and advanced through a guide slot as shown in FIG. 28, with the instrument advanced into the tibia and cutting a lateral or vertical path across a portion of the tibia.

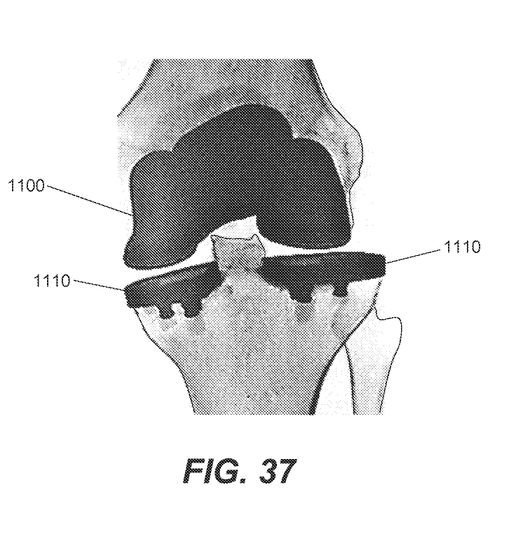

FIG. 37 shows an image of a bi-cruciate retaining patient-adapted knee replacement implant system that includes a patient-specific femoral component 1100 and patient-specific cruciate-retaining tibial tray component 1110 accommodating centrally-located soft tissue structures.

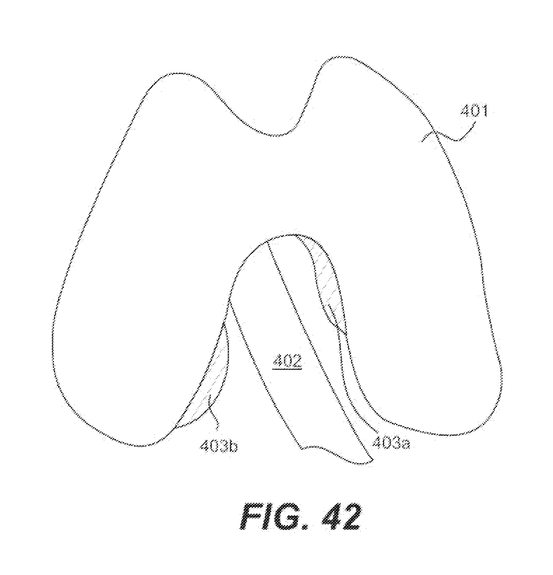

FIG. 42 is a sketch of a femoral component 401 for a bi-cruciate retaining patient-adapted knee replacement system. As shown, the intercondylar notch of the femoral component 401 is configured to accommodate the predetermined size, shape and location of the ACL 402 of the patient. The dimensions and shape of the femoral implant 401 can be designed, as shown, based on the patient's anatomy, e.g., the intercondylar notch of implant 401 substantially replicates the patient's native intercondylar notch. In a bi-cruciate retaining knee replacement system, e.g., one that includes the patient's native patella or a replaced patella (standard or patient-adapted), the patient's native notch may result in an opening wide enough for the patella to “fall” in, when the patient's knee is in deep flexion. Accordingly, a femoral component with a modified intercondylar notch can be desirable. The notch can be reshaped (e.g., having reduced width) by adding a medial portion of material 403b to implant 401 and/or adding a lateral portion of material 403a to implant 401 near the trochlea.

Accordingly, a bi-cruciate retaining patient-adapted knee replacement system can include the patient's native trochlea and native patella. Alternatively, a bi-cruciate retaining patient-adapted knee replacement system can include the patient's native trochlea and a patient-adapted patella. In certain embodiments, the patellofemoral tracking can be optimized, e.g., by providing a patient-adapted femoral component with a modified (e.g., narrower) intercondylar notch.

In various embodiments described herein, one or more features of a tibial implant component are designed and/or selected, optionally in conjunction with an implant procedure, so that the tibial implant component fits the patient. For example, in certain embodiments, one or more features of a tibial implant component and/or implant procedure are designed and/or selected, based on patient-specific data, so that the tibial implant component substantially matches (e.g., substantially negatively-matches and/or substantially positively-matches) one or more of the patient's biological structures. Alternatively or in addition, one or more features of a tibial implant component and/or implant procedure can be preoperatively engineered based on patient-specific data to provide to the patient an optimized fit with respect to one or more parameters, for example, one or more of the parameters described above. For example, in certain embodiments, an engineered bone preserving tibial implant component can be designed and/or selected based on one or more of the patient's joint dimensions as seen, for example, on a series of two-dimensional images or a three-dimensional representation generated, for example, from a CT scan or MRI scan. Alternatively or in addition, an engineered tibial implant component can be designed and/or selected, at least in part, to provide to the patient an optimized fit with respect to the engaging, joint-facing surface of a corresponding femoral implant component.

Certain embodiments include a tibial implant component having one or more patient-adapted (e.g., patient-specific or patient-engineered) features and, optionally, one or more standard features. Optionally, the one or more patient-adapted features can be designed and/or selected to fit the patient's resected tibial surface. For example, depending on the patient's anatomy and desired postoperative geometry or alignment, a patient's lateral and/or medial tibial plateaus may be resected independently and/or at different depths, for example, so that the resected surface of the lateral plateau is higher (e.g., 1 mm, greater than 1 mm, 2 mm, and/or greater than 2 mm higher) or lower (e.g., 1 mm, greater than 1 mm, 2 mm, and/or greater than 2 mm lower) than the resected surface of the medial tibial plateau.

Accordingly, in certain embodiments, tibial implant portions (i.e., medial and lateral) can be independently designed and/or selected for each of the lateral and/or medial tibial plateaus, and can then be connected (which can include an electronic or virtual modeling of the connection prior to implant manufacture and/or physically employing connection features manufactured into pre-manufactured component portions, and/or various combinations thereof) via an anterior bridge. For example, the perimeter of a lateral tibial implant component portion and the perimeter of a medial tibial implant component portion can be independently designed and/or selected to substantially match the perimeter of the resection surfaces for each of the lateral and medial tibial plateaus. If desired, the lateral tibial implant component portion and the medial tibial implant component portion can be designed using different tibial perimeter shapes, each of which substantially matches the perimeter of the corresponding resection surface, which can include tibial resection surfaces at differing depths and/or angulations or orientations with respect to the medial and lateral sections. In addition, the polyethylene layers or inserts for the lateral tibial implant component portion and the medial tibial implant component portion can have perimeter shapes that correspond to the respective implant component portion perimeter shapes. In certain embodiments, one or both of the implant components can be made entirely of a plastic or polyethylene (rather than having a polyethylene layer or insert) and each entire implant component can include a perimeter shape that substantially matches the perimeter of the corresponding resection surface. Once the individual implant component portions are designed and/or selected, an appropriate anterior bridge can be modeled, and the implant can subsequently be constructed.

Moreover, the height of a lateral tibial implant component portion and the height of a medial tibial implant component portion can be independently designed and/or selected to maintain or alter the relative heights generated by different resection surfaces for each of the lateral and medial tibial plateaus. For example, the lateral tibial implant component portion can be thicker (e.g., 1 mm, greater than 1 mm, 2 mm, and/or greater than 2 mm thicker) or thinner (e.g., 1 mm, greater than 1 mm, 2 mm, and/or greater than 2 mm thinner) than the medial tibial implant component portion to maintain or alter, as desired, the relative height of the joint-facing surface of each of the lateral and medial tibial implant components. If desired, the relative heights of the lateral and medial resection surfaces can be maintained using lateral and medial implant components portions (and lateral and medial polyethylene layers or inserts) that have the same thickness. Alternatively, the lateral implant component portion (and/or the lateral polyethylene layer or insert) can have a different thickness than the medial implant component portion (and/or the medial polyethylene layer or insert). For embodiments having one or both of the lateral and medial implant components portions made entirely of a plastic or polyethylene (rather than having a having a polyethylene layer or insert) the thickness of one implant component portion can be different from the thickness of the other implant component portion.

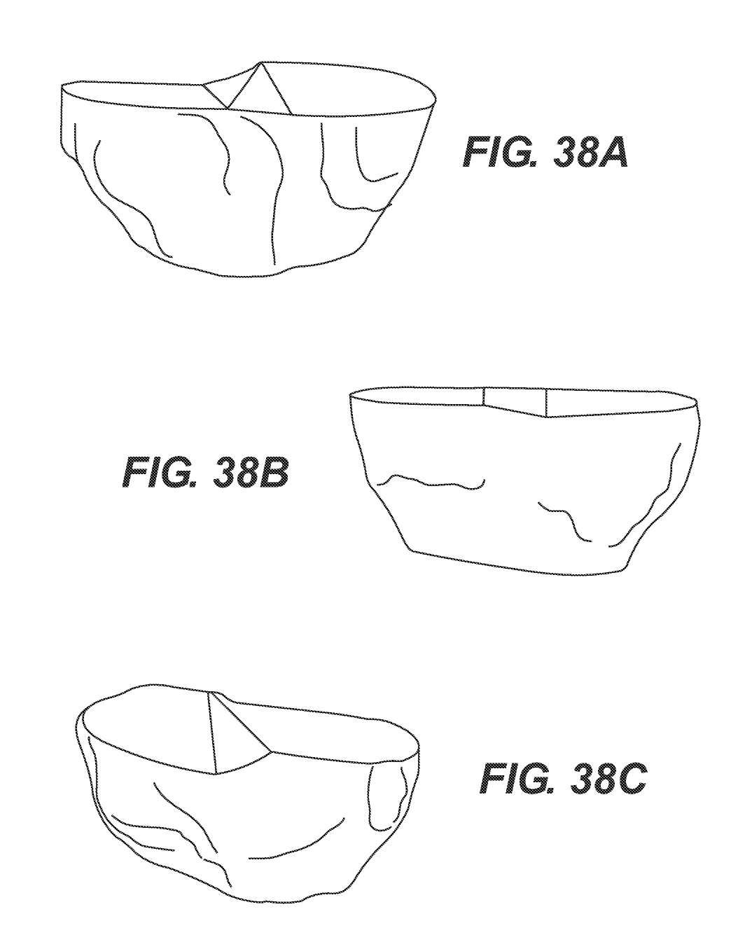

In various embodiments, different medial and lateral tibial cut heights can be accommodated and applied with a one piece tibial tray implant component, e.g., a monolithically formed, tibial tray. If desired, the tibial implant component and the corresponding resected surface of the patient's femur can have an angled surface or a step cut connecting the medial and the lateral surface facets. For example, FIGS. 38A through 38C depict three different types of step cuts separating medial and lateral resection cut facets on a patient's proximal tibia. In certain embodiments, the bone-facing surface of the tibial implant component is selected and/or designed to match these surface depths and the step cut angle, as well as other optional features such as perimeter shape. While not shown in the figures, the “step cut” surface can include a central region where anatomical structures such as the ACL and/or PCL (or other relevant structures) and/or bony support tissues have been retained and can be accommodated using a “cut out” or notch in one or more tibial tray designs, as described herein.

Tibial components also can include the same medial and lateral cut height.

In certain embodiments, the medial tibial plateau facet can be oriented at an angle different than the lateral tibial plateau facet or it can be oriented at the same angle. One or both of the medial and the lateral tibial plateau facets can be at an angle that is patient-specific, for example, similar to the original slope or slopes of the medial and/or lateral tibial plateaus, for example, in the sagittal plane. Moreover, the medial slope can be patient-specific, while the lateral slope is fixed or preset or vice versa, as exemplified herein.

### Exemplary Designs for Tibial Slopes

| MEDIAL SIDE IMPLANT SLOPE | LATERAL SIDE IMPLANT SLOPE |
|---|---|
| Patient-matched to medial plateau | Patient-matched to lateral plateau |
| Patient-matched to medial plateau | Patient-matched to medial plateau |
| Patient-matched to lateral plateau | Patient-matched to lateral plateau |
| Patient-matched to medial plateau | Not patient-matched, e.g., preset, fixed or |
| | intraoperatively adjusted |
| Patient-matched to lateral plateau | Not patient-matched, e.g., preset, fixed or |
| | intraoperatively adjusted |
| Not patient matched, e.g. preset, fixed or | Patient-matched to lateral plateau |
| intraoperatively adjusted | |
| Not patient matched, e.g., preset, fixed or | Patient-matched to medial plateau |
| intraoperatively adjusted | |
| Not patient matched, e.g. preset, fixed or | Not patient-matched, e.g. preset, fixed or |
| intraoperatively adjusted | intraoperatively adjusted |

The exemplary combinations described above can be applicable to implants that use two unicompartmental tibial inserts components with or without metal backing, one medial and one lateral. They also can be applicable to implant systems that use a single tibial implant component including all plastic designs or metal backed designs with inserts (optionally a single insert for the medial and lateral plateau, or two inserts, e.g., one medial and one lateral), for example PCL retaining, posterior stabilized, or ACL and PCL retaining implant components.

In one embodiment, an ACL and PCL (bi-cruciate retaining) total knee replacement or resurfacing device can include a tibial component with the medial implant slope matched or adapted to the patient's native medial tibial slope and a lateral implant slope matched or adapted to the patient's native lateral tibial slope. In this manner, near normal kinematics can be re-established. The tibial component can have a single metal backing component, for example with an anterior bridge connecting the medial and the lateral portion; the anterior bridge can be located anterior to the ACL. The tibial component can include two metal backed pieces (without a bridge), and/or one medial and one lateral with the corresponding plastic inserts. In the latter embodiment, a metal bridge can (or a plurality of anterior bridges can), optionally, be attachable or removable. The width of the metal bridge can be patient matched or patient adapted, e.g., matching the width of the base of the medial and lateral tibial spines or an offset added to or subtracted from this distance or a value derived from the intercondylar distance or intercondylar notch width. The width of the metal bridge can be estimated based on the ML dimension of the tibial plateau or portions thereof.

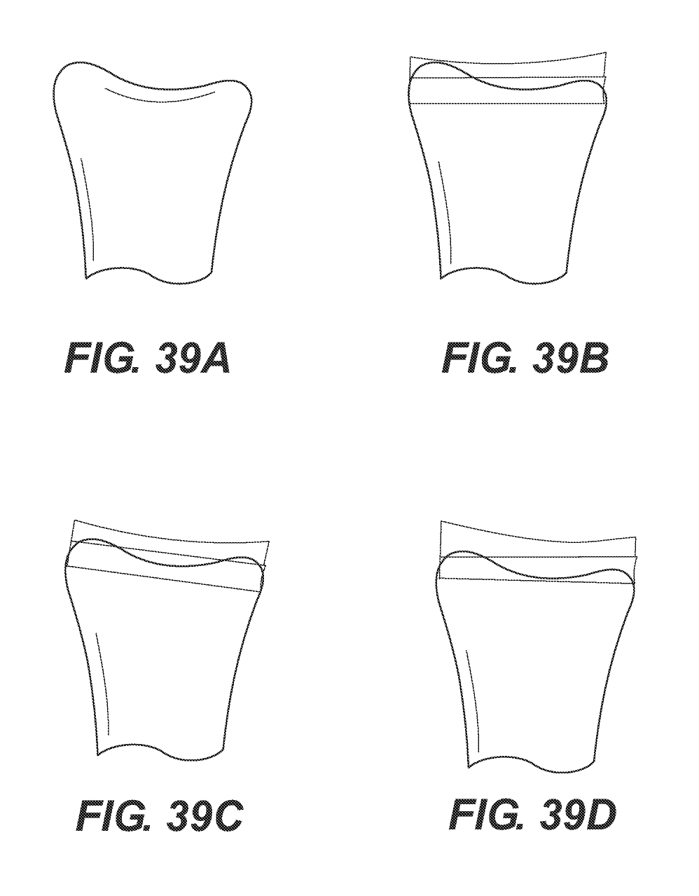

In various embodiments, the slope can be set via the alignment of the metal backed component(s). Alternatively, the metal backed component(s) can have substantially no slope in their alignment, while the medial and/or lateral slopes or both are contained or set through the insert topography or shape. One embodiment of such an implant is disclosed in FIG. 39D.

FIG. 39A depicts one embodiment of a patient's native tibial plateau in an uncut condition. FIG. 39B depicts one embodiment of an intended position of an inferior metal backed component and a superior insert. Both the metal backed component and the insert have no significant slope in this embodiment.

FIG. 39C shows one embodiment of a metal backed component wherein the bone was cut at an angle similar to the patient's slope, e.g., on the medial tibial plateau or lateral tibial plateau or, both, placing the metal backed component at a slope similar to that of the patient's native tibial plateau. The insert has no significant slope but follows the slope of the cut and the metal backed component.

FIG. 39D depicts an alternate embodiment of a metal backed component implanted with no significant slope. The tibial insert topography is, however, asymmetrical, and, in this case either selected or designed to closely approximate the patient's native tibial slope. In this example, this is achieved by selecting or designing a tibial insert that is substantially thicker anterior when compared to posterior. The difference in insert height anteriorly and posteriorly results in a slope similar to the patient's slope.

These embodiments, and derivations thereof, can be applied to a medial plateau, a lateral plateau or combinations thereof or both. In various alternative embodiments, and derivations thereof, various combinations of tilted and/or untilted inserts and/or tilted and/or untilted metal backed components or component portions can be utilized to achieve a wide variety of surgical corrections and/or account for a wide variation in patient anatomy and/or surgical cuts necessary for treating the patient. For example, where the natural slope of a patient's tibia requires a non-uniform resection (i.e., the cut portion is non-planar across the bone or is tilted and non-perpendicular relative to the mechanical axis of the bone, whether medially-laterally, anterior-posteriorly, or any combination thereof) or the surgical correction creates such a non-uniform or tilted resection, one or more correction factors can be designed into the metal backed component, into the tibial insert(s), or into any combinations thereof. Moreover, the slope can naturally or artificially be made to vary from one side of the knee to the other, or anterior to posterior, and the implant components can account for such variation.

Various of the described embodiments will be best suited for treating non-uniform or tilted natural anatomy and/or resections of partial or total knees, while others will be more appropriate for the treatment of non-uniform or tilted natural anatomy and/or resections of other joints, including a spine, spinal articulations, an intervertebral disk, a facet joint, a shoulder, an elbow, a wrist, a hand, a finger, a hip, an ankle, a foot, or a toe joint.

In various embodiments, the slope for a medial and/or lateral facet preferably is between 0 and 7 degrees, but other embodiments with other slope angles outside that range can be used. The slope can vary across one or both tibial facets from anterior to posterior. For example, a lesser slope, e.g. 0-1 degrees, can be used anteriorly, and a greater slope can be used posteriorly, for example, 4-5 degrees. Variable slopes across at least one of a medial or a lateral tibial facet can be accomplished, for example, with use of burrs (for example guided by a robot) or with use of two or more bone cuts on at least one of the tibial facets. In certain embodiments, two separate slopes can be used medially and laterally. Independent tibial slope designs can be useful for achieving bone preservation. In addition, independent slope designs can be advantageous in achieving implant kinematics that will be more natural, closer to the performance of a normal knee or the patient's knee.

In certain embodiments, the slope can be fixed, e.g. at 3, 5 or 7 degrees in the sagittal plane. In certain embodiments, the slope, either medial or lateral or both, can be patient-specific. The patient's medial slope can be used to derive the medial tibial component slope and, optionally, the lateral component slope, in either a single or a two-piece tibial implant component. The patient's lateral slope can be used to derive the lateral tibial component slope and, optionally, the medial component slope, in either a single or a two-piece tibial implant component. A patient's slope typically is between 0 and 7 degrees. In select instances, a patient may show a medial or a lateral slope that is greater than 7 degrees. In this case, if the patient's medial slope has a higher value than 7 degrees or some other pre-selected threshold, the patient's lateral slope can be applied to the medial tibial implant component portion or to the medial side of a single tibial implant component portion. If the patient's lateral slope has a higher value than 7 degrees or some other pre-selected threshold, the patient's medial slope can be applied to the lateral tibial implant component portion or to the lateral side of a single tibial implant component portion. Alternatively, if the patient's slope on one or both medial and lateral sides exceeds a pre-selected threshold value, e.g., 7 degrees or 8 degrees or 10 degrees, a fixed slope can be applied to the medial component portion or side, to the lateral component portion or side, or both. The fixed slope can be equal to the threshold value, e.g., 7 degrees or it can be a different value.

If desired, a fixed tibial slope can be used in any of the embodiments described herein.

In other embodiments, mathematical functions can be applied to derive a medial implant slope and/or a lateral implant slope, or both (wherein both can be the same). In certain embodiments, the mathematical function can include a measurement derived from one or more of the patient's joint dimensions as seen, for example, on a series of two-dimensional images or a three-dimensional representation generated, for example, from a CT scan or MRI scan. For example, the mathematical function can include a ratio between a geometric measurement of the patient's femur and the patient's tibial slope. Alternatively or in addition, the mathematical function can be or include the patient's tibial slope divided by a fixed value. In certain embodiments, the mathematical function can include a measurement derived from a corresponding implant component for the patient, for example, a femoral implant component, which itself can include patient-specific, patient-engineered, and/or standard features. Many different possibilities to derive the patient's slope using mathematical functions can be applied by someone skilled in the art.

In certain embodiments, the medial and lateral tibial plateau can be resected at the same angle. For example, a single resected cut or the same multiple resected cuts can be used across both plateaus. In other embodiments, the medial and lateral tibial plateau can be resected at different angles. Multiple resection cuts can be used when the medial and lateral tibial plateaus are resected at different angles. Optionally, the medial and the lateral tibia also can be resected at a different distance relative to the tibial plateau. In this setting, the two horizontal plane tibial cuts medially and laterally can have different slopes and/or can be accompanied by one or two vertical or oblique resection cuts, typically placed medial to the tibial plateau components.

The medial tibial implant component plateau can have a flat, convex, concave, or dished surface and/or it can have a thickness different than the lateral tibial implant component plateau. The lateral tibial implant component plateau can have a flat, convex, concave, or dished surface and/or it can have a thickness different than the medial tibial implant component plateau. The different thickness can be achieved using a different material thickness, for example, metal thickness or polyethylene or insert thickness on either side. In certain embodiments, the lateral and medial surfaces are selected and/or designed to closely resemble the patient's anatomy prior to developing the arthritic state.

The height of the medial and/or lateral tibial implant component plateau, e.g., metal only, ceramic only, metal backed with polyethylene or other insert, with single or dual inserts and single or dual tray configurations can be determined based on the patient's tibial shape, for example using an imaging test.

Alternatively, the height of the medial and/or lateral tibial component plateau, e.g. metal only, ceramic only, metal backed with polyethylene or other insert, with single or dual inserts and single or dual tray configurations, can be determined based on the patient's femoral shape. For example, if the patient's lateral condyle has a smaller radius than the medial condyle and/or is located more superior than the medial condyle with regard to its bearing surface, the height of the tibial component plateau can be adapted and/or selected to ensure an optimal articulation with the femoral bearing surface. In this example, the height of the lateral tibial component plateau can be adapted and/or selected so that it is higher than the height of the medial tibial component plateau. Since polyethylene is typically not directly visible on standard x-rays, metallic or other markers can optionally be included in the inserts in order to indicate the insert location or height, in particular when asymmetrical medial and lateral inserts or inserts of different medial and lateral thickness are used.

Alternatively, the height of the medial and/or lateral tibial component plateau, e.g. metal only, ceramic only, metal backed with polyethylene or other insert, with single or dual inserts and single or dual tray configurations can be determined based on the shape of a corresponding implant component, for example, based on the shape of certain features of the patient's femoral implant component. For example, if the femoral implant component includes a lateral condyle having a smaller radius than the medial condyle and/or is located more superior than the medial condyle with regard to its bearing surface, the height of the tibial implant component plateaus can be adapted and/or selected to ensure an optimal articulation with the bearing surface(s) of the femoral implant component. In this example, the height of the lateral tibial implant component plateau can be adapted and/or selected to be higher than the height of the medial tibial implant component plateau.

Moreover, the surface shape, e.g. mediolateral or anteroposterior curvature or both, of the tibial insert(s) can reflect the shape of the femoral component. For example, the medial insert shape can be matched to one or more radii on the medial femoral condyle of the femoral component. The lateral insert shape can be matched to one or more radii on the lateral femoral condyle of the femoral component. The lateral insert may optionally also be matched to the medial condyle. The matching can occur, for example, in the coronal plane. This has benefits for wear optimization. A pre-manufactured insert can be selected for a medial tibia that matches the medial femoral condyle radii in the coronal plane with a pre-selected ratio, e.g. 1:5 or 1:7 or 1:10. Any combination is possible. A pre-manufactured insert can be selected for a lateral tibia that matches the lateral femoral condyle radii in the coronal plane with a pre-selected ratio, e.g. 1:5 or 1:7 or 1:10. Any combination is possible. Alternatively, a lateral insert can also be matched to a medial condyle or a medial insert shape can also be matched to a lateral condyle. These combinations are possible with single and dual insert systems with metal backing. Someone skilled in the art will recognize that these matchings can also be applied to implants that use all polyethylene tibial components; i.e. the radii on all polyethylene tibial components can be matched to the femoral radii in a similar manner.

The matching of radii can also occur in the sagittal plane. For example, a cutter can be used to cut a fixed coronal curvature into a tibial insert or all polyethylene tibia that is matched to or derived from a femoral implant or patient geometry. The path and/or depth that the cutter is taking can be driven based on the femoral implant geometry or based on the patient's femoral geometry prior to the surgery. Medial and lateral sagittal geometry can be the same on the tibial inserts or all poly tibia. Alternatively, each can be cut separately. By adapting or matching the tibial poly geometry to the sagittal geometry of the femoral component or femoral condyle, a better functional result may be achieved. For example, more physiologic tibiofemoral motion and kinematics can be enabled. Alternatively, the path and/or depth that the cutter is taking can be driven based on the patient's tibial geometry prior to the surgery, optionally including estimates of meniscal shape. Medial and lateral sagittal geometry can be the same on the tibial inserts or all poly tibia. Alternatively, each can be cut separately. By adapting or matching the tibial poly geometry to the sagittal geometry of the patient's tibial plateau, a better functional result may be achieved. For example, more physiologic tibiofemoral motion and kinematics can be enabled. In the latter embodiment at least portions of the femoral sagittal J-curve can be matched to or derived from or selected based on the tibial implant geometry or the patient's tibial curvature, medially or laterally or combinations thereof.

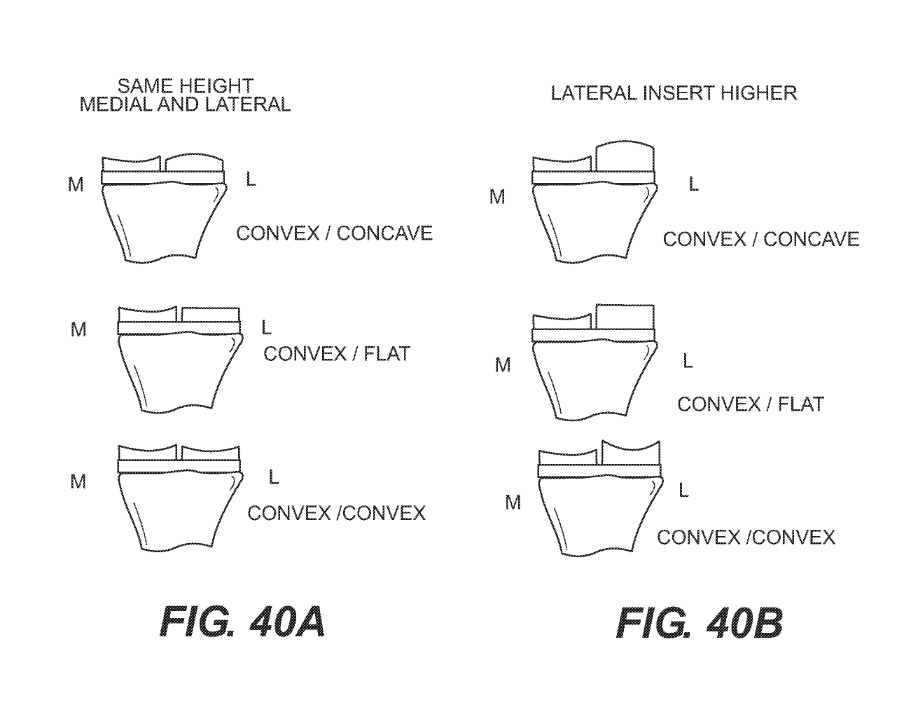

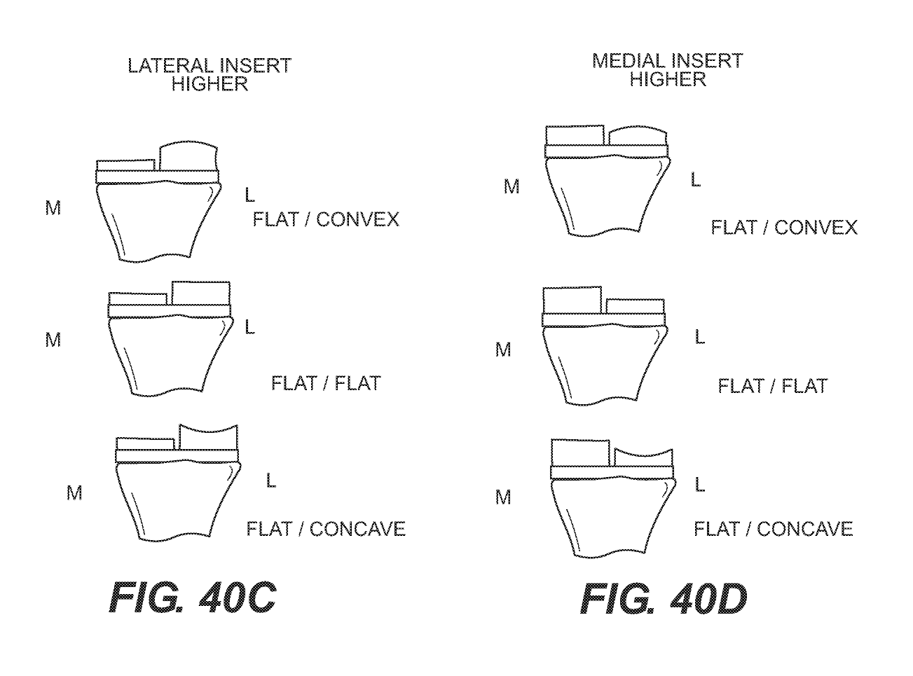

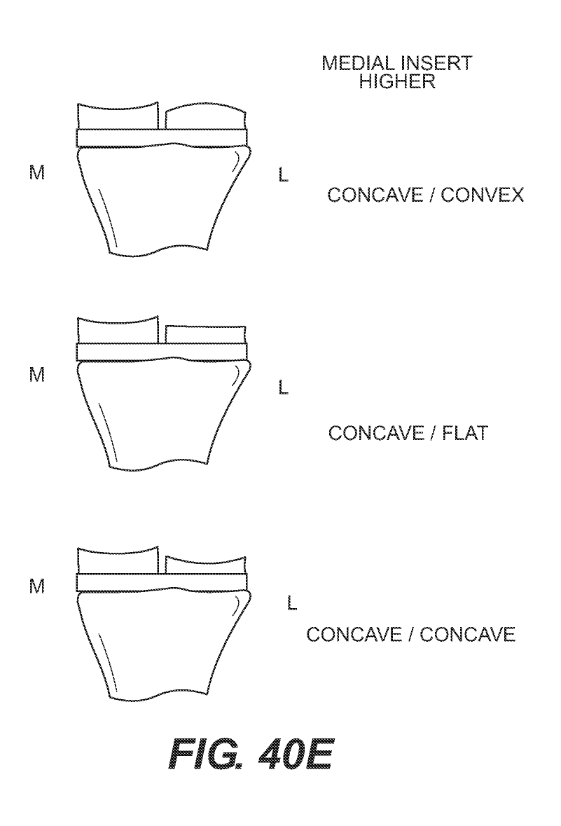

FIGS. 40A through 40E show exemplary combinations of tibial tray designs. In various embodiments, the tibial implant surface topography can be selected for, adapted to or matched to one or more femoral geometries. For example, the distance of the lowest point of the medial dish or trough to the lowest point of the lateral dish or trough can be selected from or derived from or matched to the femoral geometry, e.g. an intercondylar distance or an intercondylar notch width. In this manner, the tibial component(s) can be adapted to the femoral geometry, ensuring that the lowest point of the femoral bearing surface will mate with the lowest point of the resultant tibial bearing surface. For example, an exemplary femoral geometry may be determined or derived, and then a matching or appropriate tibial implant geometry and surface geometry can be derived from the femoral geometry (i.e., from anatomical or biomechanical or kinematic features in the sagittal and/or coronal plane of the femur) or from a combination of the femoral geometry with the tibial geometry. In such combination cases, it may be desirable to optimize the tibial implant geometry based on a weighted combination of the tibial and femoral anatomical or biomechanical or kinematic characteristics, to create a hybrid implant that accomplishes a desired correction, but which accommodates the various structural, biomechanical and/or kinematic features and/or limitations of each individual portion of the joint. In a similar manner, multi-complex joint implants having three or more component support structures, such as the knee (i.e., patella, femur and tibia), elbow (humerus, radius and ulna), wrist (radius, ulna and carpals), and ankle (fibula, tibia, talus and calcaneus) can be modeled and repaired/replaced with components modeled, derived and manufactured incorporating features of two or more mating surfaces and underlying support structures of the native joint.

The perimeter of the tibial component, metal backed, optionally poly inserts, or all plastic or other material, can be matched to or derived from the patient's tibial shape and/or the prepared tibial surface shape, and can be optimized for different cut heights and/or tibial slopes. In a preferred embodiment, the perimeter shape is matched to the cortical bone of the cut surface and the notch shape is matched to the shape of the remaining tibial structures of the central region. The surface topography of the tibial bearing surface can be designed or selected to match or reflect at least a portion of the tibial geometry, in one or more planes, e.g., a sagittal plane or a coronal plane, or both. The medial tibial implant surface topography can be selected or designed to match or reflect all or portions of the medial tibial geometry in one or more planes, e.g., sagittal and coronal. The lateral tibial implant surface topography can be selected or designed to match or reflect all or portions of the lateral tibial geometry in one or more planes, e.g., sagittal and coronal. The medial tibial implant surface topography can be selected or designed to match or reflect all or portions of the lateral tibial geometry in one or more planes, e.g., sagittal and coronal. The lateral tibial implant surface topography can be selected or designed to match or reflect all or portions of the medial tibial geometry in one or more planes, e.g., sagittal and coronal.

In various embodiments, the design and/or placement of the tibial component can be influenced (or otherwise “driven) by various factors of the femoral geometry. For example, it may be desirous to rotate the design of some or all of a tibial component (i.e., the entirety of the component and it's support structure or some portion thereof, including the tibial tray and/or the articulating poly insert and/or merely the surface orientation of the articulating surface of the tibial insert) to some degree to accommodate various features of the femoral geometry, such as the femoral epicondylar axis, posterior condylar axis, medial or lateral sagittal femoral J-curves, or other femoral axis or landmark. In a similar manner, the design and/or placement of the femoral component (i.e., the entirety of the femoral component and it's support structure or some portion thereof, including the orientation and/or placement of one or more condyles, condyle surfaces and/or the trochlear groove) can be influenced (or “driven”) by various factors of the tibial geometry, including various tibial axes, shapes, medial and/or lateral slopes and/or landmarks, e.g. tibial tuberosity, Q-angle etc. Both femoral and tibial components can be influenced in shape or orientation by the shape, dimensions, biomechanics or kinematics of the patellofemoral joint, including, for example, trochlear angle and Q-angle, sagittal trochlear geometry, coronal trochlear geometry, etc.

The surface topography of the tibial bearing surface(s) can be designed or selected to match or reflect at least portions of the femoral geometry or femoral implant geometry, in one or more planes, e.g., a sagittal plane or a coronal plane, or both. The medial implant surface topography can be selected or designed to match or reflect all or portions of the medial femoral geometry or medial femoral implant geometry in one or more planes. The lateral implant surface topography can be selected or designed to match or reflect all or portions of the lateral femoral geometry or lateral femoral implant geometry in one or more planes. The medial implant surface topography can be selected or designed to match or reflect all or portions of the lateral femoral geometry or lateral femoral implant geometry in one or more planes. The lateral implant surface topography can be selected or designed to match or reflect all or portions of the medial femoral geometry or medial femoral implant geometry in one or more planes. The medial and/or the lateral surface topography can be fixed in one, two or all dimensions. The latter can typically be used when at least one femoral geometry, e.g., the coronal curvature, is also fixed.

For example, a portion of a sagittal curvature of a femoral condyle can be used to derive and manufacture a portion of a sagittal curvature of a tibial plateau bearing surface. In one embodiment, a CNC machine can have a sagittal sweep plane through a polyethylene bearing surface that corresponds to at least a portion of a femoral sagittal curvature. The coronal radius of the cutter tool can be matched or derived from at least portions of the femoral coronal curvature or it can be a ratio or other mathematical function applied to the femoral curvature. Of note, the femoral coronal curvature can vary along the condyle allowing for smaller and larger radii in different locations. These radii can be patient specific or engineered. For example, two or more engineered radii can be applied to a single femoral condyle in two or more locations, which can be the same or different with respect to the second condyle.

If desired, a femoral bearing surface can be derived off a tibial shape in one or more dimensions using the same or similar approaches. Likewise, a femoral head or humeral head bearing surface can be derived of an acetabulum or glenoid in one or more directions or the reverse.

The implant surface topography can include one or more of the following:
Curvature of convexity in sagittal plane, optionally patient derived or matched, e.g., based on tibial or femoral geometry
Curvature of convexity in coronal plane, optionally patient derived or matched, e.g., based on tibial or femoral geometry
Curvature of concavity in sagittal plane, optionally patient derived or matched, e.g., based on tibial or femoral geometry
Curvature of concavity in coronal plane, optionally patient derived or matched, e.g., based on tibial or femoral geometry
Single sagittal radius of curvature, optionally patient derived or matched, e.g., based on tibial or femoral geometry
Multiple sagittal radii of curvature, optionally patient derived or matched, e.g., based on tibial or femoral geometry
Single coronal radius of curvature, optionally patient derived or matched, e.g., based on tibial or femoral geometry
Multiple coronal radii of curvature, optionally patient derived or matched, e.g., based on tibial or femoral geometry
Depth of dish, optionally patient derived or matched, e.g., based on tibial or femoral geometry
Depth of dish optionally adapted to presence or absence of intact anterior and/or posterior cruciate ligaments
Location of dish, optionally patient derived or matched, e.g., based on tibial or femoral geometry
AP length of dish, optionally patient derived or matched, e.g., based on tibial or femoral geometry
ML width of dish, optionally patient derived or matched, e.g., based on tibial or femoral geometry
Depth of trough, optionally patient derived or matched, e.g., based on tibial or femoral geometry
Depth of trough optionally adapted to presence or absence of intact anterior and/or posterior cruciate ligaments
Location of trough, optionally patient derived or matched, e.g., based on tibial or femoral geometry
AP length of trough, optionally patient derived or matched, e.g., based on tibial or femoral geometry
ML width of trough, optionally patient derived or matched, e.g., based on tibial or femoral geometry
Curvature of trough, optionally patient derived or matched, e.g., based on tibial or femoral geometry

All of the tibial designs discussed can be applied with a:
single piece tibial polyethylene insert, for example with a single metal backed component
single piece tibial insert of other materials, for example with a single metal backed component
two piece tibial polyethylene inserts, for example with a single metal backed component
two piece tibial inserts of other materials, for example with a single metal backed component
single piece all polyethylene tibial implant
two piece all polyethylene tibial implant, e.g. medial and lateral
single piece metal tibial implant (e.g., metal on metal or metal on ceramic)
two piece metal tibial implant, e.g., medial and lateral (e.g., metal on metal or metal on ceramic)
single piece ceramic tibial implant
two piece ceramic tibial implant, e.g., medial and lateral

Any material or material combination currently known in the art and developed in the future can be used.

Certain embodiments of tibial trays can have the following features, although other embodiments are possible: modular insert system (polymer); cast cobalt chrome; standard blanks (cobalt portion and/or modular insert) can be made in advance, then shaped patient-specific to order; thickness based on size (saves bone, optimizes strength); allowance for 1-piece or 2-piece insert systems; and/or different medial and lateral fins. In various embodiments, notch geometries can be shaped patient-specific to order.

In certain embodiments, the tibial tray is designed or cut from a blank so that the tray outer periphery matches the edge of the cut tibial bone, for example, the patient-matched peripheral geometry achieves >70%, >80%, >90%, or >95% cortical coverage. In certain embodiments, the tray periphery is designed to have substantially the same shape, but be slightly smaller, than the cortical area. In various embodiments, notch geometries are shaped to match and or accommodate (i.e., be slightly oversized relative to) remaining anatomical tibial structures.

The patient-adapted tibial implants of certain embodiments allow for design flexibility. For example, inserts can be designed to compliment an associated condyle of a corresponding femoral implant component, and can vary in dimensions to optimize design, for example, one or more of height, shape, curvature (preferably flat to concave), and location of curvature to accommodate natural or engineered wear pattern.

In the knee, a tibial cut can be selected so that it is, for example, 90 degrees perpendicular to the tibial mechanical axis or to the tibial anatomical axis. The cut can be referenced, for example, by finding the intersect with the lowest medial or lateral point on the plateau.

The slope for tibial cuts typically is between 0 and 7 or 0 and 8 degrees in the sagittal plane. Rarely, a surgeon may elect to cut the tibia at a steeper slope. The slope can be selected or designed into a patient-specific cutting jig using a preoperative imaging test. The slope can be similar to the patient's preoperative slope on at least one of a medial or one of a lateral side. The medial and lateral tibia can be cut with different slopes. The slope also can be different from the patient's preoperative slope on at least one of a medial or one of a lateral side.

The tibial cut height can differ medially and laterally, as previously described. In some patients, the uncut lateral tibia can be at a different height, for example, higher or lower, than the uncut medial tibia. In this instance, the medial and lateral tibial cuts can be placed at a constant distance from the uncut medial and the uncut lateral tibial plateau, resulting in different cut heights medially or laterally. Alternatively, they can be cut at different distances relative to the uncut medial and lateral tibial plateau, resulting in the same cut height on the remaining tibia. Alternatively, in this setting, the resultant cut height on the remaining tibia can be elected to be different medially and laterally. In certain embodiments, independent design of the medial and lateral tibial resection heights, resection slopes, and/or implant component (e.g., tibial tray and/or tibial tray insert), can enhance bone perseveration on the medial and/or lateral sides of the proximal tibia as well as on the opposing femoral condyles.

As shown in various locations in FIGS. 40A through 40E, the medial portion of a tibial implant may be higher or lower than the lateral tibial portion (or vica-versa) to compensate for different sizes of the medial and lateral femoral condyle. This method can facilitate maintenance of a patient's normal J-curve and thus help preserve normal knee kinematics. Alternatively, the effect may be achieved by offsetting the higher tibial articular surface to be the same height as the other compartment. If the condylar J-curve is offset by the same amount, the same kinematic motion can be achieved. Offsetting the femoral J-curve by the corresponding amount desirably reduces the amount of bone to be removed from the femoral condyle.

In certain embodiments, one or more patient-specific proximal tibia cuts (and the corresponding bone-facing surface of the tibial component portion(s)) is designed by: (1) finding the tibial axis perpendicular plane (“TAPP”); (2) lowering the TAPP, for example, 2 mm below the lowest point of the medial tibial plateau; (3) sloping the lowered TAPP 5 degrees posteriorly (no additional slope is required on the proximal surface of the insert); (4) fixing the component posterior slope, for example, at 5 degrees; and (5) using the tibial anatomic axis derived from Cobb or other measurement technique for tibial implant rotational alignment. If various embodiments, resection cut depths deeper than 2 mm below the lowest point of the patient's uncut medial or lateral plateau (e.g., medial plateau) may be selected and/or designed, for example, if the patient's anatomy includes an abnormality or diseased tissue below this point, or if the surgeon prefers a lower cut. For example, resection cut depths of 2.5 mm, 3 mm, 3.5 mm, 4 mm, 4.5 mm, or 5 mm can be selected and/or designed and, optionally, one or more corresponding tibial and/or femoral implant thicknesses can be selected and/or designed based on this patient-specific information.

In certain embodiments, a patient-specific proximal tibial cut portion (and the corresponding bone-facing surface of the tibial component portion) can use the preceding design except for determining the A-P slope of the cut. In certain embodiments, a patient-specific A-P slope can be used, for example, if the patient's anatomic slope is between 0 degrees and 7 degrees, or between 0 degrees and 8 degrees, or between 0 degrees and 9 degrees; a slope of 7 degrees can be used if the patient's anatomic slope is between 7 degrees and 10 degrees, and a slope of 10° can be used if the patient's anatomic slope is greater than 10 degrees.

In certain embodiments, a patient-specific A-P slope is used if the patient's anatomic slope is between 0 and 7 degrees and a slope of 7 degrees is used if the patient's anatomic slope is anything over 7 degrees. Someone skilled in the art will recognize other methods for determining the tibial slope and for adapting it during implant and jig design to achieve a desired implant slope.

A different tibial slope can be applied on the medial and the lateral side. A fixed slope can be applied on one side, while the slope on the other side can be adapted based on the patient's anatomy. For example, a medial slope can be fixed at 5 degrees, while a lateral slope matches that of the patient's tibia. In this setting, two unicondylar tibial insert portions or tray components can be used. Alternatively, a single tibial component, optionally with metal backing, can be used wherein said component does not have a flat, bone-facing surface, but includes, for example, an oblique portion to connect the medial to the lateral side substantially negatively-match resected lateral and medial tibial surfaces.

In certain embodiments, the axial profile (e.g., perimeter shape) of the tibial implant can be designed to match the axial profile of the patient's cut tibia, for example as described in U.S. Patent Application Publication No. 2009/0228113 to Lang et al, the disclosure of which is incorporated herein by reference in its entirety. Alternatively or in addition, in certain embodiments, the axial profile of the tibial implant can be designed to maintain a certain percentage or distance in its perimeter shape relative to the axial profile of the patient's cut tibia. Alternatively or in addition, in certain embodiments, the axial profile of the tibial implant can be designed to maintain a certain percentage or overhang in its perimeter shape relative to the axial profile of the patient's cut tibia. In various embodiments, the notch geometry of the tibial tray can match or accommodate the remaining tibial surface structures and/or connective tissues, such as the ACL and/or PCL.

Any of the tibial implant components described above can be derived from a blank that is cut to include one or more patient-specific features.

Tibial tray designs can include patient-specific, patient-engineered, and/or standard features. For example, in certain embodiments the tibial tray can have a front-loading design that requires minimal impaction force to seat it. The trays can come in various standard or standard blank designs, for example, small, medium and large standard or standard blank tibial trays can be provided. If desired, the tibial tray perimeters can include a blank perimeter shape that can be designed based on the design of the patient's resected proximal tibia surface. In certain embodiments, small and medium trays can include a base thickness of 2 mm (e.g., such that a patient's natural joint line may be raised 3-4 mm if the patient has 2-3 mm of cartilage on the proximal tibia prior to the disease state). Large trays can have a base thickness of 3 mm (such that in certain embodiments it may be beneficial to resect an additional 1 mm of bone so that the joint line is raised no more than 2-3 mm (assuming 2-3 mm of cartilage on the patient's proximal tibia prior to the disease state). A series of different blank sizes can also be included that accommodate differing notch sizes, shapes and/or geometries.

In various embodiments, a tibial implant design may incorporate one or more locking mechanisms to secure a tibial insert into a tibial tray. In one exemplary locking mechanism, a corresponding lower surface on the tibial insert can engage one or more ridges on the surface of the tibial tray, thereby locking the tibial insert in a desired position relative to the tray. The locking mechanism can be pre-configured and/or available, for example, in two or three different geometries or sizes. Optionally, a user or a computer program can have a library of CAD files or subroutines with different sizes and geometries of locking mechanisms available. For example, in a first step, the user or computer program can define, design or select a tibial, acetabular or glenoid implant profile that best matches a patient's cut (or, optionally, uncut) tibia, acetabulum or glenoid. In a second step, the user or computer program can then select the pre-configured CAD file or subroutine that is best suited for a given tibial or acetabular or glenoid perimeter or other shape or location or size. Moreover, the type of locking mechanism (e.g. snap, dovetail etc.) can be selected based on patient specific parameters, e.g. body weight, height, gender, race, activity level etc.).

A patient-specific peg alignment (e.g., either aligned to the patient's mechanical axis or aligned to another axis) can be combined with a patient-specific A-P cut plane. For example, in certain embodiments the peg alignment can tilt anteriorly at the same angle that the A-P slope is designed. In certain embodiments, the peg can be aligned in relation to the patient's sagittal mechanical axis, for example, at a predetermined angle relative to the patient's mechanical axis.

The joint-facing surface of a tibial implant component can include a medial bearing surface, a lateral bearing surface and an anterior bridge surface. Like femoral implant bearing surface(s), a bearing surface on a tibial implant (e.g., a groove or depression or a convex portion in the tibial surface that receives contact from a femoral component condyle) can be of standard design, for example, available in 6 or 7 different shapes, with a single radius of curvature or multiple radii of curvature in one dimension or more than one dimension. Alternatively, a bearing surface can be standardized in one or more dimensions and patient-adapted in one or more dimensions. A single radius of curvature and/or multiple radii of curvature can be selected in one dimension or multiple dimensions. Some of the radii can be patient-adapted.

Each of the two contact areas of the polyethylene insert of the tibial implant component that engage the femoral medial and lateral condyle surfaces can be any shape, for example, convex, flat, or concave, and can have any radii of curvature. In certain embodiments, any one or more of the curvatures of the medial or lateral contact areas can include patient-specific radii of curvature. Specifically, one or more of the coronal curvature of the medial contact area, the sagittal curvature of the medial contact area, the coronal curvature of the lateral contact area, and/or the sagittal curvature of the lateral contact area can include, at least in part, one or more patient-specific radii of curvature. In certain embodiments, the tibial implant component is designed to include one or both medial and lateral bearing surfaces having a sagittal curvature with, at least in part, one or more patient-specific radii of curvature and a standard coronal curvature. In certain embodiments, the bearing surfaces on one or both of the medial and lateral tibial surfaces can include radii of curvature derived from (e.g., the same length or slightly larger, such as 0-10% larger than) the radii of curvature on the corresponding femoral condyle. Having patient-adapted sagittal radii of curvature, at least in part, can help achieve normal kinematics with full range of motion.

Alternatively, the coronal curvature can be selected, for example, by choosing from a family of standard curvatures the one standard curvature having the radius of curvature or the radii of curvature that is most similar to the coronal curvature of the patient's uncut femoral condyle or that is most similar to the coronal curvature of the femoral implant component.

In preferred embodiments, one or both tibial medial and lateral contact areas have a standard concave coronal radius that is larger, for example slightly larger, for example, between 0 and 1 mm, between 0 and 2 mm, between 0 and 4 mm, between 1 and 2 mm, and/or between 2 and 4 mm larger, than the convex coronal radius on the corresponding femoral component. By using a standard or constant coronal radius on a femoral condyle with a matching standard or constant coronal radius or slightly larger on a tibial insert, for example, with a tibial radius of curvature of from about 1.05× to about 2×, or from about 1.05× to about 1.5×, or from about 1.05× to about 1.25×, or from about 1.05× to about 1.10×, or from about 1.05× to about 1.06×, or about 1.06× of the corresponding femoral implant coronal curvature. The relative convex femoral coronal curvature and slightly larger concave tibial coronal curvature can be selected and/or designed to be centered to each about the femoral condylar centers.

In the sagittal plane, one or both tibial medial and lateral concave curvatures can have a standard curvature slightly larger than the corresponding convex femoral condyle curvature, for example, between 0 and 1 mm, between 0 and 2 mm, between 0 and 4 mm, between 1 and 2 mm, and/or between 2 and 4 mm larger, than the convex sagittal radius on the corresponding femoral component. For example, the tibial radius of curvature for one or both of the medial and lateral sides can be from about 1.1× to about 2×, or from about 1.2× to about 1.5×, or from about 1.25× to about 1.4×, or from about 1.30× to about 1.35×, or about 1.32× of the corresponding femoral implant sagittal curvature. In certain embodiments, the depth of the curvature into the tibial surface material can depend on the height of the surface into the joint gap. As mentioned, the height of the medial and lateral tibial component joint-facing surfaces can be selected and/or designed independently. In certain embodiments, the medial and lateral tibial heights are selected and/or designed independently based on the patient's medial and lateral condyle height difference. In addition or alternatively, in certain embodiments, a threshold minimum or maximum tibial height and/or tibial insert thickness can be used. For example, in certain embodiments, a threshold minimum insert thickness of 6 mm is employed so that no less than a 6 mm medial tibial insert is used.

By using a tibial contact surface sagittal and/or coronal curvature selected and/or designed based on the curvature(s) of the corresponding femoral condyles or a portion thereof (e.g., the bearing portion), the kinematics and wear of the implant can be optimized. For example, this approach can enhance the wear characteristics a polyethylene tibial insert. This approach also has some manufacturing benefits. Any of the above embodiments are applicable to other joints and related implant components including an acetabulum, a femoral head, a glenoid, a humeral head, an ankle, a foot joint, an elbow including a capitellum and an olecranon and a radial head, and a wrist joint.

In various embodiments, the position and/or dimensions of anchoring and/or securement mechanisms such as a tibial implant component post or projection can be adapted based on patient-specific dimensions. For example, the post or projection can be matched with the position of the posterior cruciate ligament or the PCL insertion. It can be placed at a predefined distance from anterior or posterior cruciate ligament or ligament insertion, from the medial or lateral tibial spines or other bony or cartilaginous landmarks or sites. By matching the position of the post with the patient's anatomy, it is possible to achieve a better functional result, better replicating the patient's original anatomy.

The tray component can be machined, molded, casted, manufactured through additive techniques such as laser sintering or electron beam melting or otherwise constructed out of a metal or metal alloy such as cobalt chromium. Similarly, the insert component may be machined, molded, manufactured through rapid prototyping or additive techniques or otherwise constructed out of a plastic polymer such as ultra high molecular weight polyethylene. Other known materials, such as ceramics including ceramic coating, may be used as well, for one or both components, or in combination with the metal, metal alloy and polymer described above. It should be appreciated by those of skill in the art that an implant may be constructed as one piece out of any of the above, or other, materials, or in multiple pieces out of a combination of materials. For example, a tray component constructed of a polymer with a two-piece insert component constructed one piece out of a metal alloy and the other piece constructed out of ceramic.

Each of the components may be constructed as a “standard” or “blank” in various sizes or may be specifically formed for each patient based on their imaging data and anatomy. Computer modeling may be used and a library of virtual standards may be created for each of the components. A library of physical standards may also be amassed for each of the components.

Imaging data including shape, geometry, e.g., M-L, A-P, and S-I dimensions, then can be used to select the standard component, e.g., a femoral component or a tibial component or a humeral component and a glenoid component that most closely approximates the select features of the patient's anatomy. Typically, these components will be selected so that they are slightly larger than the patient's articular structure that will be replaced in at least one or more dimensions. The standard component is then adapted to the patient's unique anatomy, for example by removing overhanging material, e.g. using machining.

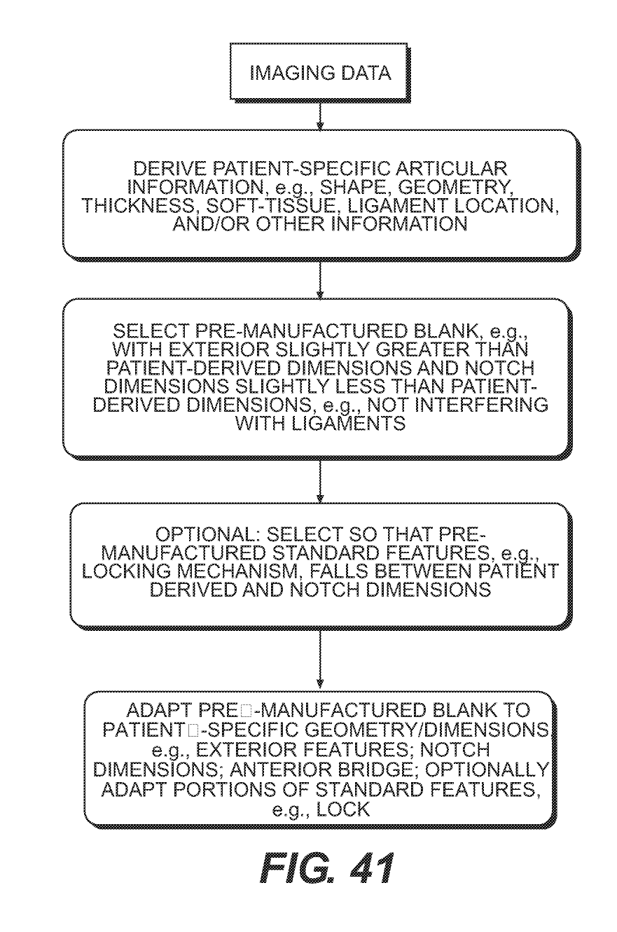

Thus, referring to the flow chart shown in FIG. 41, in a first step, the imaging data will be analyzed, either manually or with computer assistance, to determine the patient specific parameters relevant for placing the implant component. These parameters can include patient specific articular dimensions and geometry and also information about ligament location, size, and orientation, as well as potential soft-tissue impingement, and, optionally, kinematic information.

In a second step, one or more standard components, e.g., a femoral component or a tibial component or tibial insert, are selected. These are selected so that they are at least slightly greater than one or more of the derived patient specific articular dimensions and so that they can be shaped to the patient specific articular dimensions. Alternatively, these are selected so that they will not interfere with any adjacent soft-tissue structures. Combinations of both are possible.

If an implant component is used that includes an insert, e.g., a polyethylene insert and a locking mechanism in a metal or ceramic base, the locking mechanism can be adapted to the patient's specific anatomy in at least one or more dimensions. The locking mechanism can also be patient adapted in all dimensions. The location of locking features can be patient adapted while the locking feature dimensions, for example between a femoral component and a tibial component, can be fixed. Alternatively, the locking mechanism can be pre-fabricated; in this embodiment, the location and dimensions of the locking mechanism will also be considered in the selection of the pre-fabricated components, so that any adaptations to the metal or ceramic backing relative to the patient's articular anatomy do not compromise the locking mechanism. Thus, the components can be selected so that after adaptation to the patient's unique anatomy a minimum material thickness of the metal or ceramic backing will be maintained adjacent to the locking mechanism.

Since the tibia has the shape of a champagne glass, i.e., since it tapers distally from the knee joint space down, moving the tibial cut(s) (medial, lateral and anterior bridge cuts) distally will typically result in a smaller resultant cross-section of the cut tibial plateau, e.g., the ML and/or AP dimension of the cut tibia will be smaller. Typically, increasing the slope of a tibial cut will result in an elongation of the AP dimension of the cut surface—requiring a resultant elongation of a patient matched tibial component portion. Thus, in one embodiment it is possible to select an optimal standard, pre-made tibial blank for a given resection height and/or slope. This selection can involve (1) patient-adapted metal with a standard poly insert; or (2) metal and poly insert, wherein both are adapted to patient anatomy. The metal can be selected so that based on cut tibial dimensions there is always a certain minimum metal perimeter (in one, two or three dimensions) guaranteed after patient adaptation so that a lock mechanism will not fail. Optionally, one can determine minimal metal perimeter based on finite element modeling (FEA) (once during initial design of standard lock features, or patient specific every time e.g. via patient specific FEA modeling).

The tibial tray can be selected (or a metal base for other joints) to optimize percent cortical bone coverage at resection level. This selection can be (1) based on one dimension, e.g., ML; (2) based on two dimensions, e.g. ML and AP; and/or (3) based on three dimensions, e.g., ML, AP, SI or slope.

The selection can be performed to achieve a target percentage coverage of the resected bone (e.g. area) or cortical edge or margin at the resection level (e.g. AP, ML, perimeter), e.g. 85%, 90%, 95%, 98% or 100%. Optionally, a smoothing function can be applied to the derived contour of the patient's resected bone or the resultant selected, designed or adapted implant contour so that the implant does not extend into all irregularities or crevices of the virtually and then later surgically cut bone perimeter.

Optionally, a function can be included for deriving the desired implant shape that allows changing the tibial implant perimeter (either or both of the external perimeter as well as the inner notch perimeter) if the implant overhangs the cortical edge in a convex outer contour portion or in a concave outer contour portion (e.g. “crevice”). These changes can subsequently be included in the implant shape, e.g. by machining select features into the outer perimeter.

Those of skill in the art will appreciate that a combination of standard and customized components may be used in conjunction with each other. For example, a standard tray component may be used with an insert component that has been individually constructed for a specific patient based on the patient's anatomy and joint information.

Another embodiment can incorporate a tray component with one half of a two-piece insert component integrally formed with the tray component, leaving only one half of the two-piece insert to be inserted during surgery. For example, the tray component and medial side of the insert component may be integrally formed, with the lateral side of the insert component remaining to be inserted into the tray component during surgery. Of course, the reverse could also be used, wherein the lateral side of the insert component is integrally formed with the tray component leaving the medial side of the insert component for insertion during surgery.

Each of these alternatives results in a tray component and an insert component shaped so that once combined, they create a uniformly shaped implant matching the geometries of the patient's specific joint.

The step of designing an implant component and/or guide tool as described herein can include both configuring one or more features, measurements, and/or dimensions of the implant and/or guide tool (e.g., derived from patient-specific data from a particular patient and adapted for the particular patient) and manufacturing the implant. In certain embodiments, manufacturing can include making the implant component and/or guide tool from starting materials, for example, metals and/or polymers or other materials in solid (e.g., powders or blocks) or liquid form. In addition or alternatively, in certain embodiments, manufacturing can include altering (e.g., machining) an existing implant component and/or guide tool, for example, a standard blank implant component and/or guide tool or an existing implant component and/or guide tool (e.g., selected from a library). The manufacturing techniques to making or altering an implant component and/or guide tool can include any techniques known in the art today and in the future. Such techniques include, but are not limited to additive as well as subtractive methods, i.e., methods that add material, for example to a standard blank, and methods that remove material, for example from a standard blank.

In various embodiments, implant components generated by different techniques can be assessed and compared for their accuracy of shape relative to the intended shape design, for their mechanical strength, and for other factors. In this way, different manufacturing techniques can supply another consideration for achieving an implant component design with one or more target features. For example, if accuracy of shape relative to the intended shape design is critical to a particular patient's implant component design, then the manufacturing technique supplying the most accurate shape can be selected. If a minimum implant thickness is critical to a particular patient's implant component design, then the manufacturing technique supplying the highest mechanical strength and therefore allowing the most minimal implant component thickness, can be selected. Branner et al. describe a method a method for the design and optimization of additive layer manufacturing through a numerical coupled-field simulation, based on the finite element analysis (FEA). Branner's method can be used for assessing and comparing product mechanical strength generated by different additive layer manufacturing techniques, for example, SLM, DMLS, and LC.

In certain embodiments, an implant can include components and/or implant component parts produced via various methods. For example, in certain embodiments for a knee implant, the knee implant can include a metal femoral implant component produced by casting or by an additive manufacturing technique and having a patient-specific femoral intercondylar distance; a tibial component cut from a blank and machined to be patient-specific for the perimeter of the patient's cut tibia; and a tibial insert having a standard lock and a top surface that is patient-specific for at least the patient's intercondylar distance between the tibial insert dishes to accommodate the patient-specific femoral intercondylar distance of the femoral implant.

Any material known in the art can be used for any of the implant systems and component described in the foregoing embodiments, for example including, but not limited to metal, metal alloys, combinations of metals, plastic, polyethylene, cross-linked polyethylene's or polymers or plastics, pyrolytic carbon, nanotubes and carbons, as well as biologic materials.

Any fixation techniques and combinations thereof known in the art can be used for any of the implant systems and component described in the foregoing embodiments, for example including, but not limited to cementing techniques, porous coating of at least portions of an implant component, press fit techniques of at least a portion of an implant, ingrowth techniques, etc.

The above embodiments are applicable to all joints of a body, e.g., ankle, foot, elbow, hand, wrist, shoulder, hip, spine, or other joint.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

What is claimed is:

1. A tibial implant for positioning on a proximal surface of a patient's tibia, the tibial implant comprising:
an inferior surface, a superior surface, and an outer perimeter edge extending between the inferior and superior surfaces;
a notch sized and shaped to accommodate one or more anatomical structures of the patient's knee joint; and
a notch edge extending between the inferior and superior surfaces,
wherein at least a portion of the outer perimeter edge is configured to substantially match at least a corresponding portion of a periphery of the proximal surface when the tibial tray is positioned on the proximal surface.

2. A surgical tool for use in surgery on a tibia of a knee joint of a patient, comprising:
a block having a patient-specific surface, the patient-specific surface having at least a portion that is substantially a negative shape of a corresponding portion of an anterior surface of the tibia;
a guide configured to accommodate a cutting or drilling tool and having a predetermined position and orientation relative to the patient-specific surface such that, when the patient-specific surface is placed against and aligned with the corresponding portion of the anterior surface of the tibia, the guide defines a cutting or drilling trajectory extending through at least a portion of a proximal end of the tibia; and
a stop configured to prevent the cutting or drilling tool from advancing along the cutting or drilling trajectory into one or more anatomical structures of the patient's knee joint.

3. A method of making a tibial implant for a tibia of a knee joint of a patient, the method comprising:
obtaining imaging data of the knee joint, including at least a portion of the tibia of the knee joint;
deriving an outer periphery of a simulated cut surface of the tibia based, at least in part, on the imaging data;
deriving information regarding the location and/or size of one or more anatomical structures of the patient's knee joint based, at least in part, on the imaging data;
selecting a pre-manufactured blank having an exterior perimeter that is larger than the derived outer periphery and having a notch that can accommodate the location and/or size of the one or more anatomical structures; and
adapting the exterior perimeter of the selected pre-manufactured blank based, at least in part, on the derived outer periphery.

4. A method of making a surgical tool for use in surgery on a tibia of a knee joint of a patient, the method comprising:
obtaining imaging data of the knee joint, including at least a portion of the tibia of the knee joint;
deriving a shape of at least a portion of an anterior surface of the tibia based, at least in part, on the imaging data;
deriving information regarding the location and/or size of one or more anatomical structures of the patient's knee joint based, at least in part, on the imaging data; and
designing the surgical tool to include:
a block having a patient-specific surface, the patient-specific surface having at least a portion that is substantially a negative of the shape of at least a portion of the anterior surface of the tibia;
a guide configured to accommodate a cutting or drilling tool and having a predetermined position and orientation relative to the patient-specific surface such that, when the patient-specific surface is placed against and aligned with the at least a portion of the anterior surface of the tibia, the guide defines a cutting or drilling trajectory extending through at least a portion of a proximal end of the tibia; and
a stop configured to prevent the cutting or drilling tool from advancing along the cutting or drilling trajectory into the one or more anatomical structures of the patient's knee joint.

5. The implant of claim 1, the surgical tool of claim 2, the method of claim 3, or the method of claim 4, wherein the one or more anatomical structures of the patient's knee joint is selected from the group of anatomical structures consisting of the posterior cruciate ligament (PCL), the anterior cruciate ligament (ACL), the medial intercondylar tubercle, the lateral intercondylar tubercle, the PCL attachment location, the ACL attachment location, structures supporting the PCL, structures supporting the ACL, and combinations thereof.

6. The tibial implant of claim 1, wherein the tibial implant is asymmetric.

7. The tibial implant of claim 1, further comprising:
a medial section, a lateral section, and a bridge section positioned between the medial section and the lateral section,
wherein the notch is positioned between the medial section and the lateral section and is positioned posterior to the bridge section, and
wherein the bridge section comprises a width from the outer perimeter edge to the notch that is based, at least in part, on patient-specific information regarding the size and/or location of the one or more anatomical structures.

8. The tibial implant of claim 1, wherein at least a portion of the notch edge is configured at an angle with respect to the sagittal plane based, at least in part, on a planned surgical window.

9. The surgical tool of claim 2, including:
a first guide configured to accommodate a cutting or drilling tool and having a predetermined position and orientation relative to the patient-specific surface such that, when the patient-specific surface is placed against and aligned with the corresponding portion of the anterior surface of the tibia, the guide defines a cutting or drilling trajectory extending posteriorly through a medial portion of the proximal end of the tibia;
a second guide configured to accommodate a cutting or drilling tool and having a predetermined position and orientation relative to the patient-specific surface such that, when the patient-specific surface is placed against and aligned with the corresponding portion of the anterior surface of the tibia, the guide defines a cutting or drilling trajectory extending posteriorly through a lateral portion of the proximal end of the tibia; and
a third guide configured to accommodate a cutting or drilling tool and having a predetermined position and orientation relative to the patient-specific surface such that, when the patient-specific surface is placed against and aligned with the corresponding portion of the anterior surface of the tibia, the guide defines a cutting or drilling trajectory extending posteriorly through a portion of the proximal end of the tibia located between the lateral portion and the medial portion.

10. A system for treating a diseased or damaged knee joint of a patient, the knee joint including a femur, a tibia, an ACL, and a PCL, the system comprising:
the tibial implant of claim 1; and
a patient-adapted femoral implant, the femoral implant having an intercondylar notch that is sized and shaped based, at least in part, on patient-specific information to accommodate the ACL and/or PCL.
